(19) United States
(12) Patent Application Publication
(10) Pub. No.: US 2024/0023549 A1
(43) Pub. Date: Jan. 25, 2024

(54) FUNGICIDAL ARYL AMIDINES

(71) Applicant: CORTEVA AGRISCIENCE LLC, INDIANAPOLIS, IN (US)

(72) Inventors: ANN BUYSSE, CARMEL, IN (US); ERIN HANCOCK, INDIANAPOLIS, IN (US); SUSANA LOPEZ, CARMEL, IN (US); BRIAN LOY, INDIANAPOLIS, IN (US); STACY T. MEYER, ZIONSVILLE, IN (US); JEFF PETKUS, INDIANAPOLIS, IN (US); ADRIAN TLAHUEXT-ACA, INDIANAPOLIS, IN (US); WEIWEI WANG, INDIANAPOLIS, IN (US); PULAN YU, CARMEL, IN (US)

(73) Assignee: CORTEVA AGRISCIENCE LLC, INDIANAPOLIS, IN (US)

(21) Appl. No.: 18/253,822

(22) PCT Filed: Nov. 22, 2021
(86) PCT No.: PCT/US2021/060245

§ 371 Date: May 22, 2023

Related U.S. Application Data
(60) Provisional application No. 63/117,156, filed on Nov. 23, 2020.

(51) Int. Cl. A01N 37/52; C07C 257/12; C07D 213/75; C07D 213/40; C07D 295/195; C07D 217/06; C07D 215/08; C07C 323/41; C07D 333/20; C07D 209/12; C07D 317/28; C07D 317/58; C07D 233/64; C07D 491/107; C07D 209/44; A01N 43/40; A01N 43/84; A01N 43/42; A01N 43/36; A01N 43/60; A01N 43/10; A01N 43/38; A01N 43/28; A01N 43/30; A01N 43/50; A01N 43/90; A01N 43/44; C07D 205/04; C07D 211/32; A01P 3/00

(52) U.S. Cl. CPC A01N 37/52; C07C 257/12; C07D 213/75; C07D 213/40; C07D 295/195; C07D 217/06; C07D 215/08; C07C 323/41; C07D 333/20; C07D 209/12; C07D 317/28; C07D 317/58; C07D 233/64; C07D 491/107; C07D 209/44; A01N 43/40; A01N 43/84; A01N 43/42; A01N 43/36; A01N 43/60; A01N 43/10; A01N 43/38; A01N 43/28; A01N 43/30; A01N 43/50; A01N 43/90; A01N 43/44; C07D 205/04; C07D 211/32; A01P 3/00

(57) ABSTRACT

This disclosure relates to aryl amidines of Formula I and their use as fungicides.

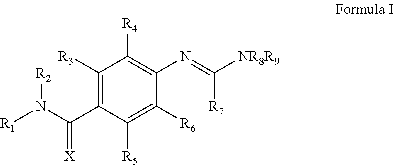

### CROSS REFERENCE TO RELATED APPLICATIONS

This application claims the benefit of U.S. Provisional Patent Application Ser. No. 63/117,156 filed Nov. 23, 2020, which is expressly incorporated by reference herein.

### BACKGROUND & SUMMARY

Fungicides are compounds, of natural or synthetic origin, which act to protect and/or cure plants against damage caused by agriculturally relevant fungi. Generally, no single fungicide is useful in all situations. Consequently, research is ongoing to produce fungicides that may have better performance, are easier to use, and cost less.

The present disclosure relates to aryl amidines and their use as fungicides. The compounds of the present disclosure may offer protection against ascomycetes, basidiomycetes, deuteromycetes and oomycetes.

One embodiment of the present disclosure may include compounds of Formula I:

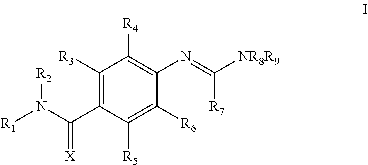

wherein
each R₁ and R₂ independently is selected from the group consisting of C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₃-C₈ cycloalkyl, C₃-C₈ substituted cycloalkyl, C₁-C₈ alkoxy, C₁-C₈ substituted alkoxy, C₃-C₈ heterocycloalkyl, C₃-C₈ substituted heterocycloalkyl, C₅-C₇ heteroaryl, C₅-C₇ substituted heteroaryl, aryl, substituted aryl, C₁-C₈ alkylaryl, substituted C₁-C₈ alkylaryl, C₁-C₈ alkyl(C₃-C₈ cycloalkyl), substituted C₁-C₈ alkyl(C₃-C₈ cycloalkyl), C₁-C₈ alkyl(C₃-C₈ heterocycloalkyl), substituted C₁-C₈ alkyl(C₃-C₈ heterocycloalkyl), C₁-C₈ alkyl(C₅-C₇ heteroaryl), and substituted C₁-C₈ alkyl(C₅-C₇ heteroaryl);
or R₁ and R₂ may be covalently bonded together to form a C₃-C₈ heterocycloalkyl, a C₃-C₈ substituted heterocycloalkyl, a C₃-C₁₂ heteroaryl, or a C₃-C₁₂ substituted heteroaryl group;
each R₃, R₄, R₅, and R₆ independently is selected from the group consisting of hydrogen, halogen, cyano, nitro, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₁-C₈ alkoxy, and C₁-C₈ substituted alkoxy;
R₇ is selected from the group consisting of hydrogen, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₁-C₈ alkoxy, C₁-C₈ substituted alkoxy, and thiol;
or R₇ and R₈ may be covalently bonded together to form a C₃-C₈ heterocycloalkyl or C₃-C₈ substituted heterocycloalkyl group;
each R₈ and R₉ independently is selected from the group consisting of C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₃-C₈ cycloalkyl, C₃-C₈ substituted cycloalkyl, aryl, substituted aryl, C₁-C₈ alkylaryl, and substituted C₁-C₈ alkylaryl;
or R₈ and R₉ may be covalently bonded together to form a saturated or unsaturated C₃-C₈ heterocycloalkyl or C₃-C₈ substituted heterocycloalkyl group;
X is selected from the group consisting of O and S; wherein any and all heterocyclic rings may contain up to three heteroatoms selected from the group consisting of O, N, and S;
or a tautomer or salt thereof.

Another embodiment of the present disclosure may include a fungicidal composition for the control or prevention of fungal attack comprising the compounds described above and a phytologically acceptable carrier material.

Yet another embodiment of the present disclosure may include a method for the control or prevention of fungal attack on a plant, the method including the steps of applying a fungicidally effective amount of one or more of the compounds described above to at least one of the fungus, a seed, the plant, and an area adjacent to the plant.

It will be understood by those skilled in the art that the following terms may include generic “R”-groups within their definitions, e.g., “the term alkoxy refers to an —OR substituent”. It is also understood that within the definitions for the following terms, these “R”-groups are included for illustration purposes and should not be construed as limiting or being limited by substitutions about Formula I.

The term “alkyl” refers to a branched, unbranched, or saturated acyclic substituent consisting of carbon and hydrogen atoms including, but not limited to, methyl, ethyl, propyl, butyl, isopropyl, isobutyl, tertiary butyl, pentyl, hexyl, and the like.

The term “alkenyl” refers to an acyclic, unsaturated (at least one carbon-carbon double bond), branched or unbranched, substituent consisting of carbon and hydrogen, including, but not limited to, ethenyl, propenyl, butenyl, isopropenyl, isobutenyl, and the like.

The term “alkynyl” refers to an acyclic, unsaturated (at least one carbon-carbon triple bond), branched or unbranched, substituent consisting of carbon and hydrogen, for example, ethynyl, propargyl, butynyl, and pentynyl.

The term “cycloalkenyl” refers to a monocyclic or polycyclic, unsaturated (at least one carbon-carbon double bond) substituent consisting of carbon and hydrogen, for example, cyclobutenyl, cyclopentenyl, cyclohexenyl, norbornenyl, bicyclo[2.2.2]octenyl, tetrahydronaphthyl, hexahydronaphthyl, and octahydronaphthyl.

The term “cycloalkyl” refers to a monocyclic or polycyclic, saturated substituent consisting of carbon and hydrogen, for example, cyclopropyl, cyclobutyl, cyclopentyl, norbornyl, bicyclo[2.2.2]octyl, and decahydronaphthyl.

The terms “aryl” and “Ar” refer to any aromatic ring, mono- or bi-cyclic, containing 0 heteroatoms, for example phenyl and naphthyl.

The terms “heteroaryl” refers to any aromatic ring, mono-, bi-cyclic, or tri-cyclic, containing 1 or more heteroatoms, for example pyridinyl, pyrimidinyl, furanyl, and thiophenyl.

The term “heterocycloalkyl” refers to any saturated, non-aromatic, mono- or bi-cyclic ring, containing carbon and hydrogen atoms and one or more heteroatoms.

The terms “alkylaryl”, “alkylheteroaryl”, “alkylcycloalkyl”, and “alkylheterocycloalkyl” refer to an alkyl group as defined herein substituted with an aryl group, a heteroaryl group, a cycloalkyl group, or a heterocycloalkyl group, respectively, as defined herein.

The term “alkoxy” refers to an —OR substituent.

The term “cyano” refers to a —C≡N substituent.

The term “amino” refers to an —N(R)₂ substituent.

The term “halogen” or “halo” refers to one or more halogen atoms, defined as F, Cl, Br, and I.

The term “nitro” refers to a —NO₂ substituent.

The term “thiol” refers to a —SH substituent.

The terms “ambient temperature” or “room temperature” refer to temperatures ranging from about 20° C. to about 24° C.

Throughout the disclosure, reference to the compounds of Formula I is read as also including all stereoisomers, for example diastereomers, enantiomers, and mixtures thereof. In another embodiment, Formula I is read as also including salts or hydrates thereof. Exemplary salts include, but are not limited to: hydrochloride, hydrobromide, hydroiodide, trifluoroacetate, and trifluoromethane sulfonate.

It is also understood by those skilled in the art that additional substitution is allowable, unless otherwise noted, as long as the rules of chemical bonding and strain energy are satisfied and the product still exhibits fungicidal activity.

Another embodiment of the present disclosure is a use of a compound of Formula I, for protection of a plant against attack by a phytopathogenic organism or the treatment of a plant infested by a phytopathogenic organism, comprising the application of a compound of Formula I, or a composition comprising the compound to soil, a plant, a part of a plant, foliage, and/or roots.

Additionally, another embodiment of the present disclosure is a composition useful for protecting a plant against attack by a phytopathogenic organism and/or treatment of a plant infested by a phytopathogenic organism comprising a compound of Formula I and a phytologically acceptable carrier material.

### DETAILED DESCRIPTION

The compounds of the present disclosure may be applied by any of a variety of known techniques, either as the compounds or as formulations comprising the compounds. For example, the compounds may be applied to the roots or foliage of plants for the control of various fungi, without damaging the commercial value of the plants. The materials may be applied in the form of any of the generally used formulation types, for example, as solutions, dusts, wettable powders, flowable concentrates, or emulsifiable concentrates.

Preferably, the compounds of the present disclosure are applied in the form of a formulation, comprising one or more of the compounds of Formula I with a phytologically acceptable carrier. Concentrated formulations may be dispersed in water, or other liquids, for application, or formulations may be dust-like or granular, which may then be applied without further treatment. The formulations can be prepared according to procedures that are conventional in the agricultural chemical art.

The present disclosure contemplates all vehicles by which one or more of the compounds may be formulated for delivery and used as a fungicide. Typically, formulations are applied as aqueous suspensions or emulsions. Such suspensions or emulsions may be produced from water-soluble, water-suspendible, or emulsifiable formulations which are solids, usually known as wettable powders; or liquids, usually known as emulsifiable concentrates, aqueous suspensions, or suspension concentrates. As will be readily appreciated, any material to which these compounds may be added may be used, provided it yields the desired utility without significant interference with the activity of these compounds as antifungal agents.

Wettable powders, which may be compacted to form water-dispersible granules, comprise an intimate mixture of one or more of the compounds of Formula I, an inert carrier and surfactants. The concentration of the compound in the wettable powder may be from about 10 percent to about 90 percent by weight based on the total weight of the wettable powder, more preferably about 25 weight percent to about 75 weight percent. In the preparation of wettable powder formulations, the compounds may be compounded with any finely divided solid, such as prophyllite, talc, chalk, gypsum, Fuller's earth, bentonite, attapulgite, starch, casein, gluten, montmorillonite clays, diatomaceous earths, purified silicates or the like. In such operations, the finely divided carrier and surfactants are typically blended with the compound(s) and milled.

Emulsifiable concentrates of the compounds of Formula I may comprise a convenient concentration, such as from about 1 weight percent to about 50 weight percent of the compound, in a suitable liquid, based on the total weight of the concentrate. The compounds may be dissolved in an inert carrier, which is either a water-miscible solvent or a mixture of water-immiscible organic solvents, and emulsifiers. The concentrates may be diluted with water and oil to form spray mixtures in the form of oil-in-water emulsions. Useful organic solvents include aromatics, especially the high-boiling naphthalenic and olefinic portions of petroleum such as heavy aromatic naphtha. Other organic solvents may also be used, for example, terpenic solvents, including rosin derivatives, aliphatic ketones, such as cyclohexanone, and complex alcohols, such as 2-ethoxyethanol.

Emulsifiers which may be advantageously employed herein may be readily determined by those skilled in the art and include various nonionic, anionic, cationic and amphoteric emulsifiers, or a blend of two or more emulsifiers. Examples of nonionic emulsifiers useful in preparing the emulsifiable concentrates include the polyalkylene glycol ethers and condensation products of alkyl and aryl phenols, aliphatic alcohols, aliphatic amines or fatty acids with ethylene oxide, propylene oxides such as the ethoxylated alkyl phenols and carboxylic esters solubilized with the polyol or polyoxyalkylene. Cationic emulsifiers include quaternary ammonium compounds and fatty amine salts. Anionic emulsifiers include the oil-soluble salts (e.g., calcium) of alkylaryl sulfonic acids, oil-soluble salts or sulfated polyglycol ethers and appropriate salts of phosphated-polyglycol ether.

Representative organic liquids which may be employed in preparing the emulsifiable concentrates of the compounds of the present disclosure are the aromatic liquids such as xylene, propyl benzene fractions, or mixed naphthalene fractions, mineral oils, substituted aromatic organic liquids such as dioctyl phthalate; kerosene; dialkyl amides of various fatty acids, particularly the dimethyl amides of fatty glycols and glycol derivatives such as the n-butyl ether, ethyl ether or methyl ether of diethylene glycol, the methyl ether of triethylene glycol, petroleum fractions or hydrocarbons such as mineral oil, aromatic solvents, paraffinic oils, and the like; vegetable oils such as soybean oil, rapeseed oil, olive oil, castor oil, sunflower seed oil, coconut oil, corn oil, cottonseed oil, linseed oil, palm oil, peanut oil, safflower oil, sesame oil, tung oil and the like; esters of the above vegetable oils; and the like. Mixtures of two or more organic liquids may also be employed in the preparation of the emulsifiable concentrate. Organic liquids include xylene, and propyl benzene fractions, with xylene being most preferred in some cases. Surface-active dispersing agents are typically employed in liquid formulations and in an amount of from 0.1 to 20 percent by weight based on the combined weight of the dispersing agent with one or more of the compounds. The formulations can also contain other compatible additives, for example, plant growth regulators and other biologically active compounds used in agriculture.

Aqueous suspensions comprise suspensions of one or more water-insoluble compounds of Formula I, dispersed in an aqueous vehicle at a concentration in the range from about 1 to about 50 weight percent, based on the total weight of the aqueous suspension. Suspensions are prepared by finely grinding one or more of the compounds, and vigorously mixing the ground material into a vehicle comprised of water and surfactants chosen from the same types discussed above. Other components, such as inorganic salts and synthetic or natural gums, may also be added to increase the density and viscosity of the aqueous vehicle.

The compounds of Formula I can also be applied as granular formulations, which are particularly useful for applications to the soil. Granular formulations generally contain from about 0.5 to about 10 weight percent, based on the total weight of the granular formulation of the compound(s), dispersed in an inert carrier which consists entirely or in large part of coarsely divided inert material such as attapulgite, bentonite, diatomite, clay or a similar inexpensive substance. Such formulations are usually prepared by dissolving the compounds in a suitable solvent and applying it to a granular carrier which has been preformed to the appropriate particle size, in the range of from about 0.5 to about 3 millimeters (mm). A suitable solvent is a solvent in which the compound is substantially or completely soluble. Such formulations may also be prepared by making a dough or paste of the carrier and the compound and solvent, and crushing and drying to obtain the desired granular particle.

Dusts containing the compounds of Formula I may be prepared by intimately mixing one or more of the compounds in powdered form with a suitable dusty agricultural carrier, such as, for example, kaolin clay, ground volcanic rock, and the like. Dusts can suitably contain from about 1 to about 10 weight percent of the compounds, based on the total weight of the dust.

The formulations may additionally contain adjuvant surfactants to enhance deposition, wetting, and penetration of the compounds onto the target crop and organism. These adjuvant surfactants may optionally be employed as a component of the formulation or as a tank mix. The amount of adjuvant surfactant will typically vary from 0.01 to 1.0 percent by volume, based on a spray-volume of water, preferably 0.05 to 0.5 volume percent. Suitable adjuvant surfactants include, but are not limited to ethoxylated nonyl phenols, ethoxylated synthetic or natural alcohols, salts of the esters or sulfosuccinic acids, ethoxylated organosilicones, ethoxylated fatty amines, blends of surfactants with mineral or vegetable oils, crop oil concentrate (mineral oil (85%)+emulsifiers (15%)); nonylphenol ethoxylate; benzylcocoalkyldimethyl quaternary ammonium salt; blend of petroleum hydrocarbon, alkyl esters, organic acid, and anionic surfactant; C₉-C₁₁ alkylpolyglycoside; phosphated alcohol ethoxylate; natural primary alcohol (C₁₂-C₁₆) ethoxylate; di-sec-butylphenol EO-PO block copolymer; polysiloxane-methyl cap; nonylphenol ethoxylate+urea ammonium nitrate; emulsified methylated seed oil; tridecyl alcohol (synthetic) ethoxylate (8EO); tallow amine ethoxylate (15 EO); PEG (400) dioleate-99. The formulations may also include oil-in-water emulsions such as those disclosed in U.S. patent application Ser. No. 11/495,228, the disclosure of which is expressly incorporated by reference herein.

Another embodiment of the present disclosure is a method for the control or prevention of fungal attack. This method comprises applying to the soil, plant, roots, foliage, or locus of the fungus, or to a locus in which the infestation is to be prevented (for example applying to cereal or grape plants), a fungicidally effective amount of one or more of the compounds of Formula I. The compounds are suitable for treatment of various plants at fungicidal levels, while exhibiting low phytotoxicity. The compounds may be useful both in a protectant and/or an eradicant fashion.

The compounds have been found to have significant fungicidal effect particularly for agricultural use. Many of the compounds are particularly effective for use with agricultural crops and horticultural plants.

It will be understood by those skilled in the art that the efficacy of the compound for the foregoing fungi establishes the general utility of the compounds as fungicides.

The compounds have broad ranges of activity against fungal pathogens. Exemplary pathogens may include, but are not limited to, the causative agent of Septoria leaf blotch of wheat (Zymoseptoria tritici), spot blotch of barley (Cochliobolus sativus), wheat brown rust (Puccinia triticina), wheat stripe rust (Puccinia striiformis), scab of apple (Venturia inaequalis), blister smut of maize (Ustilago maydis), powdery mildew of grapevine (Uncinula necator), leaf blotch of barley (Rhynchosporium commune), blast of rice (Magnaporthe grisea), Asian soybean rust (Phakopsora pachyrhizi), glume blotch of wheat (Parastagonospora nodorum), Anthracnose of cucurbits (Glomerella lagenarium), leaf spot of beet (Cercospora beticola), early blight of tomato (Alternaria solani), net blotch of barley (Pyrenophora teres), powdery mildew of wheat (Blumeria graminis f. sp. tritici), powdery mildew of barley (Blumeria graminis f. sp. hordei), powdery mildew of cucurbits (Erysiphe cichoracearum), sudden death syndrome of soybean (Fusarium virguliforme), collar rot or damping-off of seedlings (Rhizoctonia solani), root rot (Pythium ultimum), grey mold (Botrytis cinerea), Ramularia leaf spot (Ramularia collo-cygni), tan spot of wheat (Pyrenophora tritici-repentis), Northern leaf blight of maize (Exserohilum turcicum), Southern rust of maize (Puccinia polysora), white mold (Sclerotinia sclerotiorum), powdery mildew of soybean (Erysiphe diffusa), head blight of cereals (Fusarium graminearum), powdery mildew of apple (Podosphaera leucotricha), Anthracnose of soybean (Colletotrichum truncatum), Cercospora leaf blight (Cercospora kikuchii), frogeye leaf spot (Cerospora sojina), target spot of soybean (Corynespora cassiicola), and leaf spot of soybean (Septoria glycines). The exact amount of the active material to be applied is dependent not only on the specific active material being applied, but also on the particular action desired, the fungal species to be controlled, and the stage of growth thereof, as well as the part of the plant or other product to be contacted with the compound. Thus, all the compounds, and formulations containing the same, may not be equally effective at similar concentrations or against the same fungal species.

The compounds are effective in use with plants in a disease-inhibiting and phytologically acceptable amount. The term “disease-inhibiting and phytologically acceptable amount” refers to an amount of a compound that kills or inhibits the plant disease for which control is desired, but is not significantly toxic to the plant. This amount will generally be from about 0.1 to about 1000 ppm (parts per million), with 1 to 500 ppm being preferred. The exact concentration of compound required varies with the fungal disease to be controlled, the type of formulation employed, the method of application, the particular plant species, climate conditions, and the like. A suitable application rate is typically in the range from about 0.10 to about 4 pounds per acre (about 0.01 to 0.45 grams per square meter, g/m 2).

Any range or desired value given herein may be extended or altered without losing the effects sought, as is apparent to the skilled person for an understanding of the teachings herein.

The compounds of Formula I may be made using well-known chemical procedures. Intermediates not specifically mentioned in this disclosure are either commercially available, may be made by routes disclosed in the chemical literature, or may be readily synthesized from commercial starting materials utilizing standard procedures.

### General Schemes

The following schemes illustrate approaches to generating aryl amidine compounds of Formula I. The following descriptions and examples are provided for illustrative purposes and should not be construed as limiting in terms of substituents or substitution patterns.

Compounds of Formula 1.4, wherein R₃, R₄, R₅ and R₆ are as originally defined, can be prepared by the method shown in Scheme 1, steps a-c. Compounds of Formula 1.2, wherein R₃, R₅ and R₆ are as originally defined, can be prepared by the method shown in Scheme 1, step a. The compound of Formula 1.1, wherein R₃, R₅ and R₆ are as originally defined, can be treated with sodium periodate, in the presence of iodine (I₂), in a solvent, such as N,N-dimethylformamide (DMF), at a temperature from about ambient temperature to about 50° C., to afford compounds of Formula 1.2, wherein R₃, R₅ and R₆ are as originally defined, as shown in step a. Compounds of Formula 1.3, wherein R₃, R₄, R₅ and R₆ are as originally defined, can be prepared by the method shown in Scheme 1, step b. The compound of Formula 1.2, wherein R₃, R₅ and R₆ are as originally defined, can be treated with a catalyst, such as [1,1′-bis(diphenylphosphino)ferrocene]dichloropalladium(II) complex with dichloromethane (PdCl₂(dppf)DCM), and a boronic anhydride, such as B₃O₃(R₄)₃, wherein R₄ is as originally defined, in the presence of a base, such as cesium carbonate (C_{S2}CO₃), in a solvent, such as 1,4-dioxane, at a temperature from about ambient temperature to about 120° C., under microwave irradiation, to afford compounds of Formula 1.3, wherein R₃, R₄, R₅ and R₆ are as originally defined, as shown in step b. Compounds of Formula 1.4, wherein R₃, R₄, R₅ and R₆ are as originally defined, can be prepared by the method shown in Scheme 1, step c. The compound of Formula 1.3, wherein R₃, R₄, R₅ and R₆ are as originally defined, can be treated with a base, such as lithium hydroxide hydrate (LiOH·H₂O), in a solvent mixture, such as 3:2:1 tetrahydrofuran (THF):methanol (MeOH):water, at a temperature from about ambient temperature to about reflux (˜70° C.), to afford compounds of Formula 1.4, wherein R₃, R₄, R₅ and R₆ are as originally defined, as shown in step c.

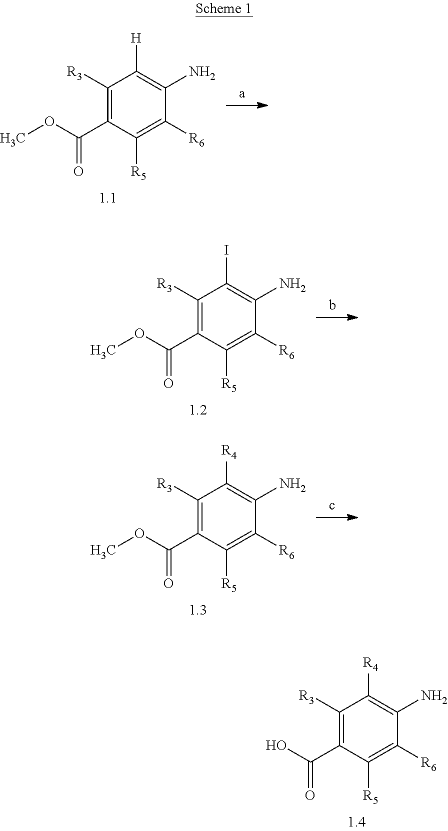

Alternatively, compounds of Formula 1.4, wherein R₃, R₄, R₅ and R₆ are as originally defined, can be prepared by the method shown in Scheme 2, steps d-f Compounds of Formula 2.2, wherein R₃, R₅ and R₆ are as originally defined, can be prepared by the method shown in Scheme 2, step d. The compound of Formula 2.1, wherein R₃, R₅ and R₆ are as originally defined, can be treated with a halogenating reagent, such as N-bromosuccinimide (NB S), in a solvent, such as DMF, at a temperature from about 0° C. to about ambient temperature, to afford compounds of Formula 2.2, wherein R₃, R₅ and R₆ are as originally defined, as shown in step d. Compounds of Formula 2.3, wherein R₃, R₄, R₅ and R₆ are as originally defined, can be prepared by the method shown in Scheme 2, step e. The compound of Formula 2.2, wherein R₃, R₅ and R₆ are as originally defined, can be treated with a catalyst, such as (2-dicyclohexylphosphino-2′,4′,6′-triisopropyl-1,1′-biphenyl)[2-(2′-amino-1,1′-biphenyl)]palladium(II) methanesulfonate (XPhos-Pd-G3), and a boronic anhydride, such as B₃O₃(R₄)₃, wherein R₄ is as originally defined, in the presence of a base, such as potassium phosphate tribasic (K₃PO₄), in a solvent mixture, such as 10:1 1,4-dioxane:water, at a temperature from about ambient temperature to about 100° C., to afford compounds of Formula 2.3, wherein R₃, R₄, R₅ and R₆ are as originally defined, as shown in step e. Compounds of Formula 1.4, wherein R₃, R₄, R₅ and R₆ are as originally defined, can be prepared by the method shown in Scheme 2, step f. The compound of Formula 2.3, wherein R₃, R₄, R₅ and R₆ are as originally defined, can be treated with a base, such as potassium hydroxide (KOH), in a solvent, such as water, at a temperature from about ambient temperature to about 60° C., to afford compounds of Formula 1.4, wherein R₃, R₄, R₅ and R₆ are as originally defined, as shown in step f

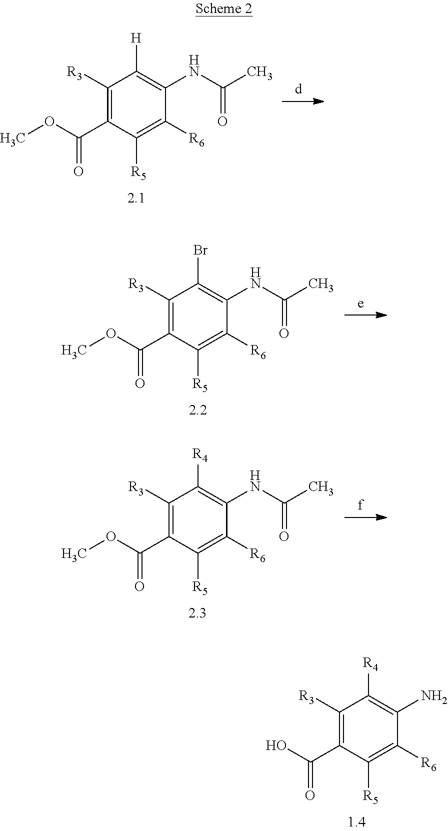

Alternatively, compounds of Formula 1.4, wherein R₃, R₄, R₅ and R₆ are as originally defined, can be prepared by the method shown in Scheme 3, steps g-n. Compounds of Formula 3.2, wherein R₃, R₄, R₅ and R₆ are as originally defined, can be prepared by the method shown in Scheme 3, step g. The compound of Formula 3.1, wherein R₃, R₄, R₅ and R₆ are as originally defined, can be treated with hydrogen bromide (HBr), in the presence of sodium nitrite (NaNO₂), in a solvent, such as acetic acid, at a temperature from about ambient temperature to about 85° C., to afford compounds of Formula 3.2, wherein R₃, R₄, R₅ and R₆ are as originally defined, as shown in step g. Compounds of Formula 3.3, wherein R₃, R₄, R₅ and R₆ are as originally defined, can be prepared by the method shown in Scheme 3, step h. The compound of Formula 3.2, wherein R₃, R₄, R₅ and R₆ are as originally defined, can be treated with a metal reagent, such as iron (Fe(0)), in the presence of an ammonium salt, such as ammonium chloride (NH₄Cl), in a solvent mixture, such as 1:1 ethanol (EtOH):water, at a temperature from about ambient temperature to about 70° C., to afford compounds of Formula 3.3, wherein R₃, R₄, R₅ and R₆ are as originally defined, as shown in step h. Alternatively, compounds of Formula 3.3, wherein R₃, R₄, R₅ and R₆ are as originally defined, can be prepared by the method shown in Scheme 3, step i. The compound of Formula 3.4, wherein R₃, R₄, R₅ and R₆ are as originally defined, can be treated with a halogenating reagent, such as NBS, in a solvent, such as DMF, at a temperature from about 0° C. to about ambient temperature, to afford compounds of Formula 3.3, wherein R₃, R₄, R₅ and R₆ are as originally defined, as shown in step i. Compounds of Formula 3.5, wherein R₃, R₄, R₅ and R₆ are as originally defined, can be prepared by the method shown in Scheme 3, step j. The compound of Formula 3.3, wherein R₃, R₄, R₅ and R₆ are as originally defined, can be treated with a metal cyanide, such as copper(I) cyanide (CuCN), in a solvent, such as N-methyl-2-pyrrolidone (NMP), at a temperature from about ambient temperature to about 180° C., under microwave irradiation, to afford compounds of Formula 3.5, wherein R₃, R₄, R₅ and R₆ are as originally defined, as shown in step j. Alternatively, compounds of Formula 3.5, wherein R₃, R₄, R₅ and R₆ are as originally defined, can be prepared by the method shown in Scheme 3, step k. The compound of Formula 3.3, wherein R₃, R₄, R₅ and R₆ are as originally defined, can be treated with a metal cyanide, such as zinc(II) cyanide (Zn(CN)₂), in the presence of a metal catalyst, such as tetrakis(triphenylphosphine)-palladium(0) (Pd(PPh₃)₄), in a solvent, such as DMF, at a temperature from about ambient temperature to about 120° C., to afford compounds of Formula 3.5, wherein R₃, R₄, R₅ and R₆ are as originally defined, as shown in step k. Compounds of Formula 1.4, wherein R₃, R₄, R₅ and R₆ are as originally defined, can be prepared by the method shown in Scheme 3, step 1. The compound of Formula 3.5, wherein R₃, R₄, R₅ and R₆ are as originally defined, can be treated with a base, such as potassium hydroxide (KOH), in a solvent, such as H₂O, at a temperature from about ambient temperature to about 120° C., to afford compounds of Formula 1.4, wherein R₃, R₄, R₅ and R₆ are as originally defined, as shown in step 1. Compounds of Formula 3.6, wherein R₃, R₄, R₅ and R₆ are as originally defined, can be prepared by the method shown in Scheme 3, step m. The compound of Formula 3.3, wherein R₃, R₄, R₅ and R₆ are as originally defined, can be treated with carbon monoxide (CO) gas in the presence of a metal catalyst, such as palladium(II) acetate (Pd(OAc)₂), in the presence of a ligand, such as 1,4-bis(diphenylphosphino)butane (dppb), with a base, such as triethylamine (TEA), in a solvent, such as MeOH, at a pressure of about 400 pounds per square inch (psi; 2758 kilopascals (kPa)) and a temperature from about ambient temperature to about 125° C., to afford compounds of Formula 3.6, wherein R₃, R₄, R₅ and R₆ are as originally defined, as shown in step m. Compounds of Formula 1.4, wherein R₃, R₄, R₅ and R₆ are as originally defined, can be prepared by the method shown in Scheme 3, step n. The compound of Formula 3.6, wherein R₃, R₄, R₅ and R₆ are as originally defined, can be treated with a base, such as lithium hydroxide hydrate (LiOH·H₂O), in a solvent mixture, such as 3:2:1 THF:MeOH:water, at a temperature from about ambient temperature to about 125° C., to afford compounds of Formula 1.4, wherein R₃, R₄, R₅ and R₆ are as originally defined, as shown in step n.

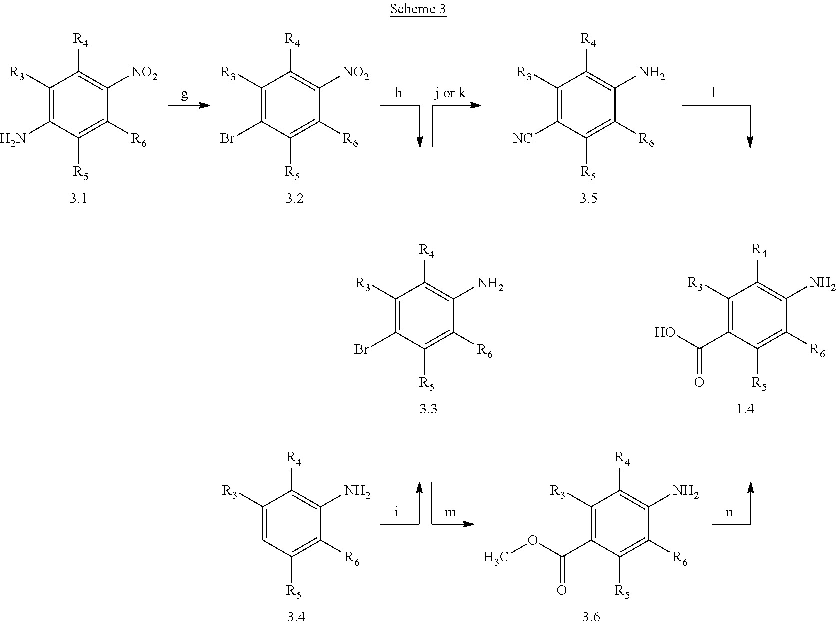

Compounds of Formula 4.2, wherein R₁, R₂, R₃, R₄, R₅ and R₆ are as originally defined, can be prepared by the method shown in Scheme 4, step o. Compounds of Formula 1.4, wherein R₃, R₄, R₅ and R₆ are as originally defined, can be treated with a secondary amine, such as a compound of Formula 4.1, wherein R₁ and R₂ are as originally defined, in the presence of a peptide coupling reagent, such as 1-ethyl-3-(3-dimethylaminopropyl)carbodiimide hydrochloride (EDCI), N,N′-dicyclohexylcarbodiimide (DCC) or benzotriazol-1-yl-oxytripyrrolidinophosphonium hexafluorophosphate (PyBOP), and a catalyst, such as 4-dimethylaminopyridine (DMAP) or N,N-diisopropylethylamine (DIPEA), in a solvent, such as dichloromethane (DCM), at a temperature from about 0° C. to about ambient temperature, to afford compounds of Formula 4.2, wherein R₁, R₂, R₃, R₄, R₅ and R₆ are as originally defined, as shown in step o.

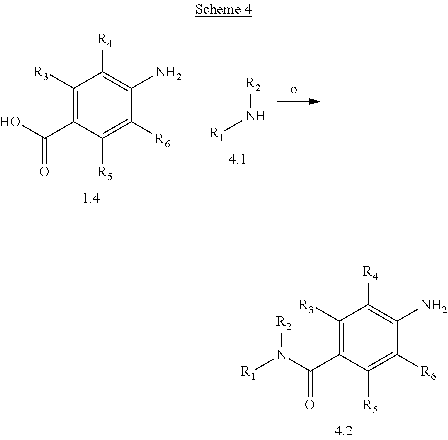

Alternatively, compounds of Formula 4.2, wherein R₁, R₂, R₃, R₄, R₅ and R₆ are as originally defined, can be prepared by the method shown in Scheme 5, steps p-q. Compounds of Formula 5.2, wherein R₁, R₃, R₄, R₅ and R₆ are as originally defined, can be prepared by the method shown in Scheme 5, step p. The compound of Formula 1.4, wherein R₃, R₄, R₅ and R₆ are as originally defined, can be treated with a primary amine, such as a compound of Formula 5.1, wherein R₁ is as originally defined, in the presence of a peptide coupling reagent, such as 1-ethyl-3-(3-dimethylaminopropyl)carbodiimide hydrochloride (EDCI) or N,N′-dicyclohexylcarbodiimide (DCC), and an activator, such as 1H-benzo[d][1,2,3]triazol-1-ol (HOBt), and a base, such as 4-dimethylaminopyridine (DMAP) or N,N-diisopropylethylamine (DIPEA), in a solvent, such as DMF, at a temperature from about 0° C. to about ambient temperature, to afford compounds of Formula 5.2, wherein R₁, R₃, R₄, R₅ and R₆ are as originally defined, as shown in step p. Compounds of Formula 4.2, wherein R₁, R₂, R₃, R₄, R₅ and R₆ are as originally defined, can be prepared by the method shown in Scheme 5, step q. The compound of Formula 5.2, wherein R₁, R₃, R₄, R₅ and R₆ are as originally defined, can be treated with a base, such as lithium bis(trimethylsilyl)amide (LHMDS), and an alkylating reagent, such as R₂—Y, wherein R₂ is as originally defined, and Y is a leaving group, such as Br or I, in a solvent, such as tetrahydrofuran (THF), at a temperature from about 0° C. to about ambient temperature, to afford compounds of Formula 4.2, wherein R₁, R₂, R₃, R₄, R₅ and R₆ are as originally defined, as shown in step q.

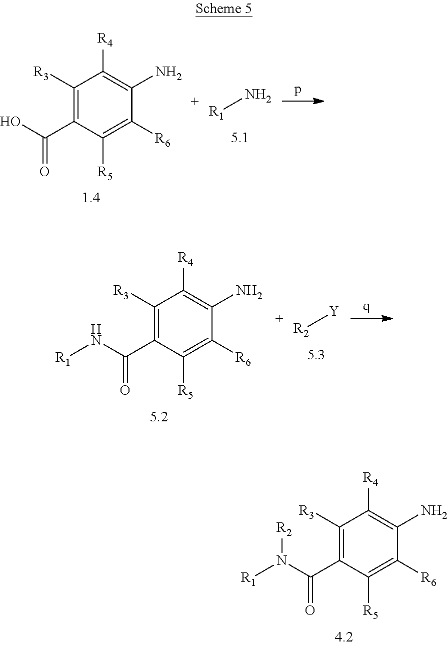

Compounds of Formula 6.2, wherein R₁, R₂, R₃, R₄, R₅, R₆, Its and R₉ are as originally defined, can be prepared by the method shown in Scheme 6, step r. The compound of Formula 4.2, wherein R₁, R₂, R₃, R₄, R₅ and R₆ are as originally defined, can be treated with an amine, such as a compound of Formula 6.1, wherein R₈ and R₉ are as originally defined, in a solvent, such as toluene, at a temperature from about ambient temperature to about reflux (˜111° C.), to afford compounds of Formula 6.2, wherein R₁, R₂, R₃, R₄, R₅, R₆, R₈ and R₉ are as originally defined, as shown in step r.

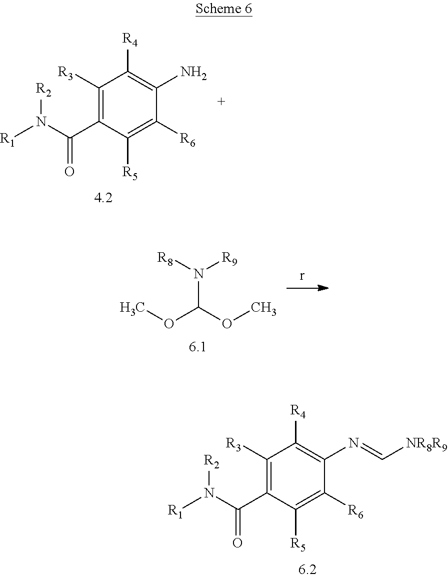

Alternatively, compounds of Formula 6.2, wherein R₁, R₂, R₃, R₄, R₅, R₆, R₈ and R₉ are as originally defined, can be prepared by the method shown in Scheme 7, steps s-t. Compounds of Formula 7.1, wherein R₁, R₂, R₃, R₄, R₅ and R₆ are as originally defined, and Z is an alkyl group, can be prepared by the method shown in Scheme 7, step s. The compound of Formula 4.2, wherein R₁, R₂, R₃, R₄, R₅ and R₆ are as originally defined, can be treated with a trialkyl orthoformate (CH(OZ)₃), such as trimethyl orthoformate or triethyl orthoformate, in the presence of an acid catalyst, such as p-toluenesulfonic acid monohydrate (pTsOH·H₂O), at a temperature from about ambient temperature to about reflux (˜100° C. or ˜140° C., respectively), to afford compounds of Formula 7.1, wherein R₁, R₂, R₃, R₄, R₅ and R₆ are as originally defined, and Z is an alkyl group, as shown in step s. Compounds of Formula 6.2, wherein R₁, R₂, R₃, R₄, R₅, R₆, R₈ and R₉ are as originally defined, can be prepared by the method shown in Scheme 7, step t. The compound of Formula 7.1, wherein R₁, R₂, R₃, R₄, R₅ and R₆ are as originally defined, and Z is an alkyl group, can be treated with an amine, such as a compound of Formula 7.2, wherein R₈ and R₉ are as originally defined, in a solvent, such as DCM, at a temperature from about ambient temperature to about reflux (˜40° C.), to afford compounds of Formula 6.2, wherein R₁, R₂, R₃, R₄, R₅, R₆, R₅ and R₉ are as originally defined, as shown in step t.

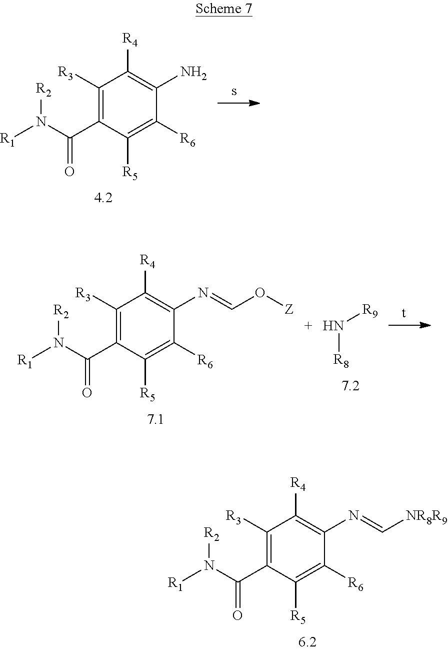

Compounds of Formula 8.2, wherein R₁, R₂, R₃, R₄, R₅, R₆, R₇, R₈ and R₉ are as originally defined, can be prepared by the method shown in Scheme 8, step u. The compound of Formula 4.2, wherein R₁, R₂, R₃, R₄, R₅ and R₆ are as previously defined, can be treated with an amide, such as a compound of Formula 8.1, wherein R₇, R₈ and R₉ are as originally defined, in the presence of a dehydrating reagent, such as oxalyl chloride ((COCl)₂) or phosphoryl trichloride (POCl₃), in a solvent, such as DCM or toluene, at a temperature from about ambient temperature to about reflux (˜40° C. or ˜111° C., respectively), to afford compounds of Formula 8.2, wherein R₁, R₂, R₃, R₄, R₅, R₆, R₇, R₈ and R₉ are as originally defined, as shown in step u.

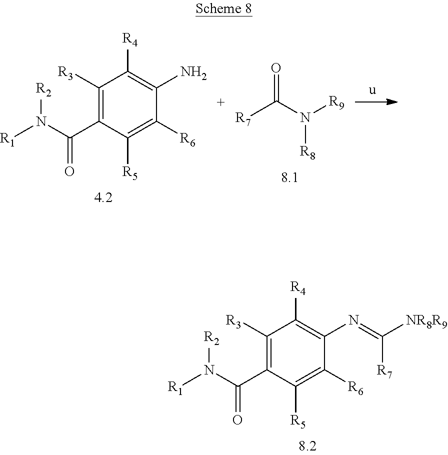

Compounds of Formula 9.4, wherein R₃, R₄, R₅, R₆, R₈ and R₉ are as originally defined, can be prepared by the method shown in Scheme 9, steps v-y. Compounds of Formula 9.1, wherein R₃, R₄, R₅ and R₆ are as originally defined, can be prepared by the method shown in Scheme 9, step v. The compound of Formula 1.4, wherein R₃, R₄, R₅ and R₆ are as previously defined, can be treated with a benzylating agent, such as benzyl bromide or benzyl chloride, in the presence of a base, such as potassium carbonate, in a solvent, such as DMF, at a temperature from about 0° C. to about ambient temperature, to afford compounds of Formula 9.1, wherein R₃, R₄, R₅ and R₆ are as originally defined, as shown in step v. Compounds of Formula 9.2, wherein R₃, R₄, R₅ and R₆ are as originally defined, and Z is an alkyl group, can be prepared by the method shown in Scheme 9, step w. The compound of Formula 9.1, wherein R₃, R₄, R₅ and R₆ are as originally defined, can be treated with a trialkyl orthoformate (CH(OZ)₃), such as trimethyl orthoformate or triethyl orthoformate, in the presence of an acid catalyst, such asp-toluenesulfonic acid monohydrate (pTsOH·H₂O), at a temperature from about ambient temperature to about reflux (˜100° C. or ˜140° C., respectively), to afford compounds of Formula 9.2, wherein R₃, R₄, R₅ and R₆ are as originally defined, and Z is an alkyl group, as shown in step w. Compounds of Formula 9.3, wherein R₃, R₄, R₅, R₆, R₈ and R₉ are as originally defined, can be prepared by the method shown in Scheme 9, step x. The compound of Formula 9.2, wherein R₃, R₄, R₅ and R₆ are as originally defined, and Z is an alkyl group, can be treated with an amine, such as a compound of Formula 7.2, wherein R₈ and R₉ are as originally defined, in a solvent, such as DCM, at a temperature from about ambient temperature to about reflux (˜40° C.), to afford compounds of Formula 9.3, wherein R₃, R₄, R₅, R₆, R₈ and R₉ are as originally defined, as shown in step x. Compounds of Formula 9.4, wherein R₃, R₄, R₅, R₆, R₈ and R₉ are as originally defined, can be prepared by the method shown in Scheme 9, step y. The compound of Formula 9.3, wherein R₃, R₄, R₅, R₆, R₈ and R₉ are as originally defined, can be treated with a metal catalyst, such as palladium on carbon (Pd/C), in the presence of a hydrogen source, such as hydrogen gas (H₂) or cyclohexene, in a solvent, such as ethyl acetate (EtOAc), at a temperature from about ambient temperature (H₂ gas) to about reflux (˜70° C., cyclohexene), to afford compounds of Formula 9.4, wherein R₃, R₄, R₅, R₆, Its and R₉ are as originally defined, as shown in step y.

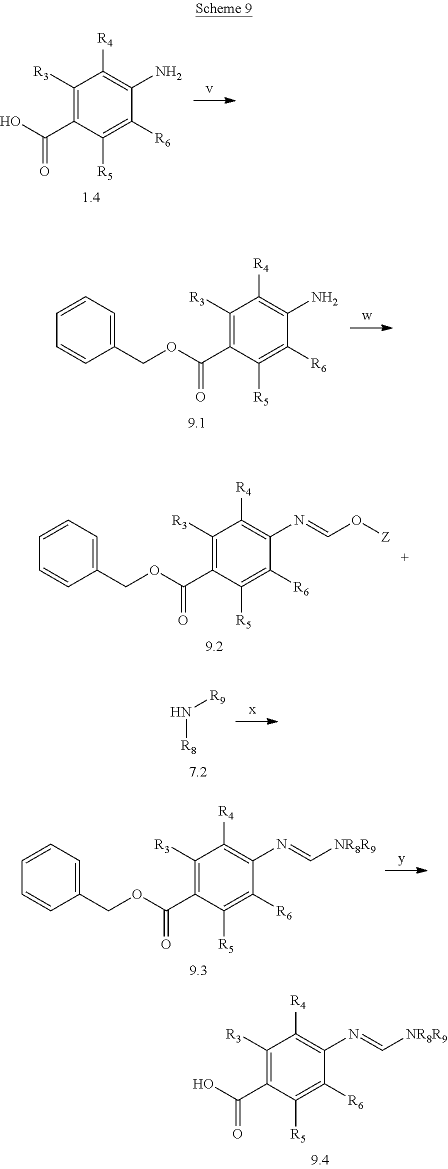

Alternatively, compounds of Formula 6.2, wherein R₁, R₂, R₃, R₄, R₅, R₆, R₈ and R₉ are as originally defined, can be prepared by the method shown in Scheme 10, step z. The compound of Formula 9.4, wherein R₃, R₄, R₅, R₆, R₈ and R₉ are as previously defined, can be treated with an amine, such as a compound of Formula 4.1, wherein R₁ and R₂ are as originally defined, in the presence of a peptide coupling reagent, such as 1-ethyl-3-(3-dimethylaminopropyl)carbodiimide hydrochloride (EDCI) or N,N′-dicyclohexylcarbodiimide (DCC), and an activator, such as 1H-benzo[d][1,2,3]triazol-1-ol (HOBt), and a base, such as 4-dimethylaminopyridine (DMAP) or N,N-diisopropylethylamine (DIPEA), in a solvent, such as DMF, at a temperature from about 0° C. to about ambient temperature, to afford compounds of Formula 6.2, wherein R₁, R₂, R₃, R₄, R₅, R₆, R₈ and R₉ are as previously defined, as shown in step z.

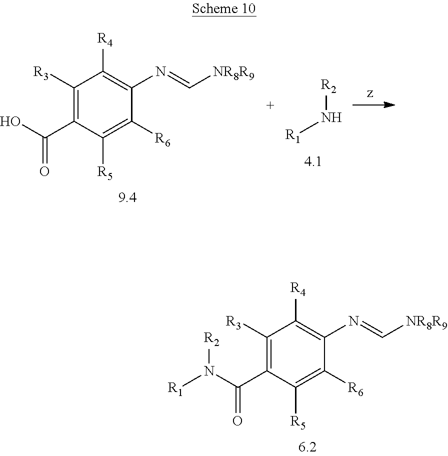

Compounds of Formula 11.4, wherein R₂ is as originally defined and R₁ is a benzyl or substituted benzyl, can be prepared by the method shown in Scheme 11, steps aa-cc. Compounds of Formula 11.4, wherein R₂ is as originally defined and R₁ is a benzyl or substituted benzyl, can be prepared by the method shown in Scheme 11, step aa. The compound of Formula 11.1, wherein the phenyl ring may be optionally substituted at any of the positions on the ring, can be treated with an amine, such as a compound of Formula 11.2, wherein R₂ is as originally defined, in the presence of a hydride source, such as sodium cyanoborohydride, and an acid, such as acetic acid, in a solvent, such as MeOH, at a temperature from about 0° C. to about ambient temperature, to afford compounds of Formula 11.4, wherein R₂ is as originally defined and R₁ is a benzyl or substituted benzyl, as shown in step aa. Alternatively, compounds of Formula 11.4, wherein R₂ is as originally defined and R₁ is a benzyl or substituted benzyl, can be prepared by the method shown in Scheme 11, steps bb-cc. Compounds of Formula 11.3, wherein R₂ is as originally defined and the phenyl ring may be optionally substituted at any of the positions on the ring, can be prepared by the method shown in Scheme 11, step bb. The compound of Formula 11.1, wherein the phenyl ring may be optionally substituted at any of the positions on the ring, can be treated with an amine, such as a compound of Formula 11.2, wherein R₂ is as originally defined, in the presence of a base, such as pyridine, in a solvent, such as MeOH, at a temperature of about ambient temperature, to afford compounds of Formula 11.3, wherein R₂ is as originally defined and the phenyl ring may be optionally substituted at any of the positions on the ring, as shown in step bb. Compounds of Formula 11.4, wherein R₂ is as originally defined and R₁ is a benzyl or substituted benzyl, can be prepared by the method shown in Scheme 11, step cc. The compound of Formula 11.3, wherein R₂ is as originally defined and the phenyl ring may be optionally substituted at any of the positions on the ring, can be treated with a hydride source, such as sodium cyanoborohydride, in the presence of an indicator, such as sodium (E)-4-((4-(dimethylamino)phenyl)diazenyl)benzenesulfonate, and an acid, such as hydrochloric acid, in a solvent, such as MeOH, at a temperature of about ambient temperature, to afford compounds of Formula 11.4, wherein R₂ is as originally defined and R₁ is a benzyl or substituted benzyl, as shown in step cc.

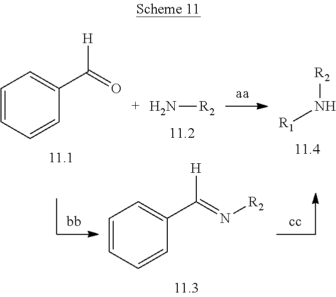

Alternatively, compounds of Formula 4.1, wherein R₁ and R₂ are as originally defined, can be prepared by the method shown in Scheme 12, steps dd-ff. Compounds of Formula 12.1, wherein R₁ is as originally defined, can be prepared by the method shown in Scheme 12, step dd.

The compound of Formula 5.1, wherein R₁ is as originally defined, can be treated with a dicarbonate, such as di-tert-butyl dicarbonate, in the presence of a base, such as triethylamine, in a solvent, such as DCM, at a temperature of about ambient temperature, to afford compounds of Formula 12.1, wherein R₁ is as originally defined, as shown in step dd. Compounds of Formula 12.2, wherein R₁ and R₂ are as originally defined, can be prepared by the method shown in Scheme 12, step ee. The compound of Formula 12.1, wherein R₁ is as originally defined, can be treated with a base, such as lithium bis(trimethylsilyl)amide (LHMDS), and an alkylating reagent, such as a compound of Formula 5.3, wherein R₂ is as originally defined, and Y is a leaving group, such as Br or I, in a solvent, such as tetrahydrofuran (THF), at a temperature from about 0° C. to about ambient temperature, to afford compounds of Formula 12.2, wherein R₁ and R₂ are as originally defined, as shown in step ee. Compounds of Formula 4.1, wherein R₁ and R₂ are as originally defined, can be prepared by the method shown in Scheme 12, step ff. The compound of Formula 12.2, wherein R₁ and R₂ are as originally defined, can be treated with an acid, such as 4 molar (M) hydrochloric acid in 1,4-dioxane, in a solvent, such as THF, at a temperature from about ambient temperature to 65° C., to afford compounds of Formula 4.1, wherein R₁ and R₂, are as originally defined, as shown in step ff.

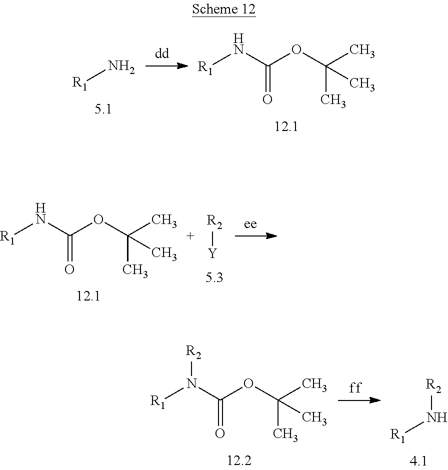

Alternatively, compounds of Formula 8.2, wherein R₁, R₂, R₃, R₄, R₅, R₆, R₇, R₈ and R₉ are as originally defined, can be prepared by the method shown in Scheme 13, steps gg-hh. Compounds of Formula 13.1, wherein R₃, R₄, R₅, R₆, R₈ and R₉ are as originally defined, can be prepared by the method shown in Scheme 13, step gg. The compound of Formula 1.4, wherein R₃, R₄, R₅ and R₆ are as previously defined, can be treated with an amide, such as a compound of Formula 8.1, wherein R₇, R₈ and R₉ are as originally defined, in the presence of a dehydrating reagent, such as oxalyl chloride ((COCl)₂), in a solvent, such as DCM, at a temperature from about ambient temperature to about reflux (˜40° C.), to afford compounds of Formula 13.1, wherein R₃, R₄, R₅, R₆, R₈ and R₉ are as previously defined, as shown in step gg. Compounds of Formula 8.2, wherein R₁, R₂, R₃, R₄, R₅, R₆, R₇, R₈ and R₉ are as originally defined, can be prepared by the method shown in Scheme 13, step hh. The compound of Formula 13.1, wherein R₃, R₄, R₅, R₆, R₅ and R₉ are as previously defined, can be treated with an amine, such as a compound of Formula 4.1, wherein R₁ and R₂ are as originally defined, in the presence of a peptide coupling reagent, such as 1-ethyl-3-(3-dimethylaminopropyl)carbodiimide hydrochloride (EDCI), and a catalyst, such as 4-dimethylaminopyridine (DMAP), and a base, such as diisopropylethylamine (DIPEA) or triethylamine (Et₃N), in a solvent, such as DCM, at a temperature from about 0° C. to about ambient temperature, to afford compounds of Formula 8.2, wherein R₁, R₂, R₃, R₄, R₅, R₆, R₇, R₈ and R₉ are as previously defined, as shown in step hh.

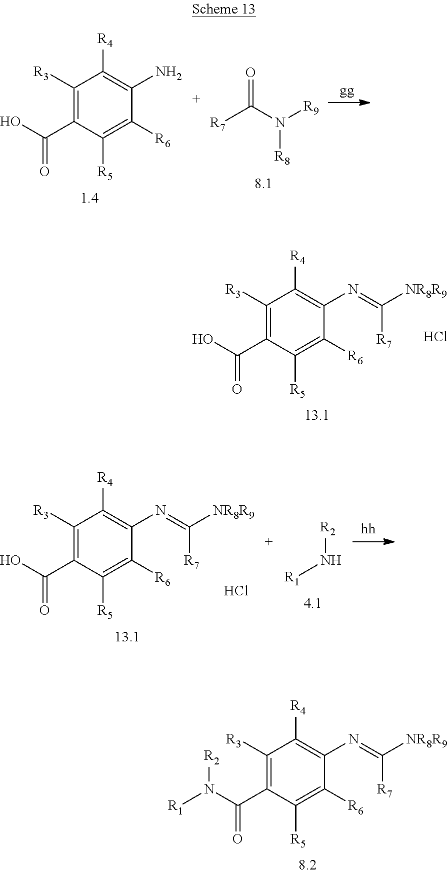

Alternatively, compounds of Formula 8.2, wherein R₁, R₂, R₃, R₄, R₅, R₆, R₇, R₅ and R₉ are as originally defined, can be prepared by the method shown in Scheme 14, steps ii-kk. Compounds of Formula 14.1, wherein R₃, R₄, R₅, R₆, R₈ and R₉ are as originally defined, can be prepared by the method shown in Scheme 14, step ii. The compound of Formula 3.3, wherein R₃, R₄, R₅ and R₆ are as previously defined, can be treated with an amide, such as a compound of Formula 8.1, wherein R₇, R₈ and R₉ are as originally defined, in the presence of a dehydrating reagent, such as oxalyl chloride ((COCl)₂), in a solvent, such as DCM, at a temperature from about 0° C. to about reflux (˜40° C.), to afford compounds of Formula 14.1, wherein R₃, R₄, R₅, R₆, R₅ and R₉ are as previously defined, as shown in step ii. Compounds of Formula 13.1, wherein R₃, R₄, R₅, R₆, R₈ and R₉ are as originally defined, can be prepared by the method shown in Scheme 14, step jj. The compound of Formula 14.1, wherein R₃, R₄, R₅, R₆, R₈ and R₉ are as previously defined, can be treated with a base, such as n-butyllithium, in the presence of a carbon dioxide (CO₂) source, such as anhydrous dry ice, in a solvent, such as THF, at a temperature from about −78° C. to about ambient temperature, followed by treatment with 4 M hydrogen chloride in 1,4-dioxane, to afford compounds of Formula 13.1, wherein R₃, R₄, R₅, R₆, R₇, R₈ and R₉ are as previously defined, as shown in step jj. Compounds of Formula 8.2, wherein R₁, R₂, R₃, R₄, R₅, R₆, R₈ and R₉ are as originally defined, can be prepared by the method shown in Scheme 14, step kk. The compound of Formula 13.1, wherein R₃, R₄, R₅, R₆, R₈ and R₉ are as previously defined, can be treated with an amine, such as a compound of Formula 4.1, wherein R₁ and R₂ are as originally defined, in the presence of a peptide coupling reagent, such as 1-ethyl-3-(3-dimethylaminopropyl)carbodiimide hydrochloride (EDCI), and a base, such as 4-dimethylaminopyridine (DMAP), in a solvent, such as DCM, at a temperature from about 0° C. to about ambient temperature, to afford compounds of Formula 8.2, wherein R₁, R₂, R₃, R₄, R₅, R₆, R₇, R₈ and R₉ are as previously defined, as shown in step kk.

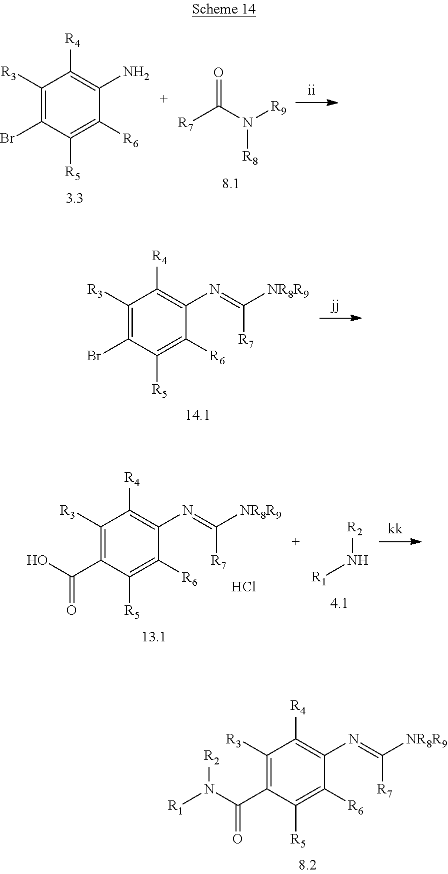

Alternatively, compounds of Formula 6.2, wherein R₁, R₂, R₃, R₄, R₅, R₆, R₈ and R₉ are as originally defined, can be prepared by the method shown in Scheme 15, steps ll-mm. Compounds of Formula 15.1, wherein R₃, R₄, R₅, R₆, R₈ and R₉ are as originally defined, can be prepared by the method shown in Scheme 15, step FL The compound of Formula 9.4, wherein R₃, R₄, R₅, R₆, R₈ and R₉ are as previously defined, can be treated with an amine, such as imidazole, in the presence of a peptide coupling reagent, such as 1-ethyl-3-(3-dimethylaminopropyl)carbodiimide hydrochloride (EDCI), and a catalyst, such as 4-dimethylaminopyridine (DMAP), in the presence of a base, such as triethylamine, in a solvent, such as DCM, at a temperature from about 0° C. to about ambient temperature, to afford compounds of Formula 15.1, wherein R₃, R₄, R₅, R₆, R₈ and R₉ are as previously defined, as shown in step ll. Compounds of Formula 6.2, wherein R₁, R₂, R₃, R₄, R₅, R₆, R₈ and R₉ are as originally defined, can be prepared by the method shown in Scheme 15, step mm. The compound of Formula 15.1, wherein R₃, R₄, R₅, R₆, R₈ and R₉ are as previously defined, can be treated with an amine, such as a compound of Formula 4.1, wherein R₁ and R₂ are as originally defined, in the presence of a base, such as 1,8-diazabicyclo[5.4.0]undec-7-ene (DBU), in a solvent, such as acetonitrile, at a temperature from about 0° C. to about ambient temperature, to afford compounds of Formula 6.2, wherein R₁, R₂, R₃, R₄, R₅, R₆, R₈ and R₉ are as previously defined, as shown in step mm.

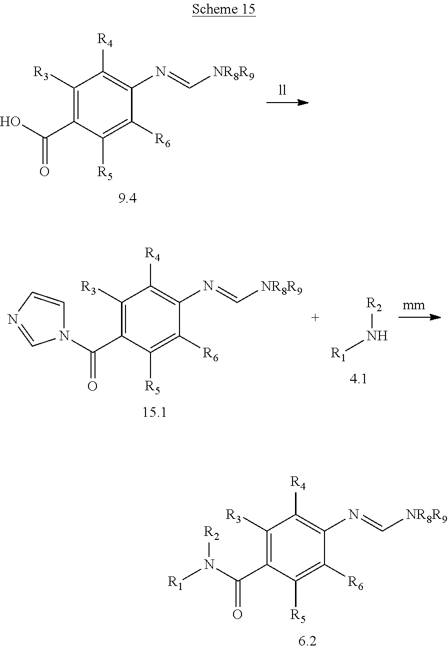

Compounds of Formula 16.4, wherein R₁, R₃, R₄, R₅ and R₆ are as originally defined, and R₂ is trifluoromethyl, can be prepared by the method shown in Scheme 16, steps nn-pp. Compounds of Formula 16.2, wherein R₁, R₃, R₄, R₅ and R₆ are as originally defined, can be prepared by the method shown in Scheme 16, step nn. The compound of Formula 16.1, wherein R₃, R₄, R₅, and R₆ are as previously defined, can be treated with a primary amine, such as a compound of Formula 5.1, wherein R₁ is as originally defined, in the presence of a peptide coupling reagent, such as 1-ethyl-3-(3-dimethylaminopropyl)carbodiimide hydrochloride (EDCI), and a catalyst, such as 4-dimethylaminopyridine (DMAP), in a solvent, such as DCM, at a temperature from about 0° C. to about ambient temperature, to afford compounds of Formula 16.2, wherein R₁, R₃, R₄, R₅ and R₆ are as originally defined, as shown in step nn. Compounds of Formula 16.3, wherein R₁, R₃, R₄, R₅ and R₆ are as originally defined, and R₂ is a trifluoromethyl group, can be prepared by the method shown in Scheme 16, step oo. The compound of Formula 16.2, wherein R₁, R₃, R₄, R₅ and R₆ are as originally defined, can be treated with a trifluoromethylating agent, such as trifluoromethanesulfonate, in the presence of a fluoride source, such as cesium fluoride, and a fluorinating agent, such as 1-(chloromethyl)-4-fluoro-1,4-diazabicyclo[2.2.2]octane-1,4-diium tetrafluoroborate (Selectfluor®), in the presence of a promoter, such as silver(I) trifluoromethanesulfonate, in a solvent mixture, such as 3:1 DCM:phenyl chloride, at a temperature of about ambient temperature, under inert atmosphere to afford compounds of Formula 16.3, wherein R₁, R₃, R₄, R₅ and R₆ are as originally defined, and R₂ is a trifluoromethyl group, as shown in step oo. Compounds of Formula 16.4, wherein R₁, R₃, R₄, R₅ and R₆ are as originally defined, and R₂ is a trifluoromethyl group, can be prepared by the method shown in Scheme 16, step pp. The compound of Formula 16.3, wherein R₁, R₃, R₄, R₅ and R₆ are as originally defined, and R₂ is a trifluoromethyl group, can be treated with a reductant, such as hydrogen gas (H₂), in the presence of a catalyst, such as 5% palladium on carbon, in a solvent, such as EtOAc, at a temperature of about ambient temperature, to afford compounds of Formula 16.4, wherein R₁, R₃, R₄, R₅ and R₆ are as originally defined, and R₂ is a trifluoromethyl group, as shown in step pp.

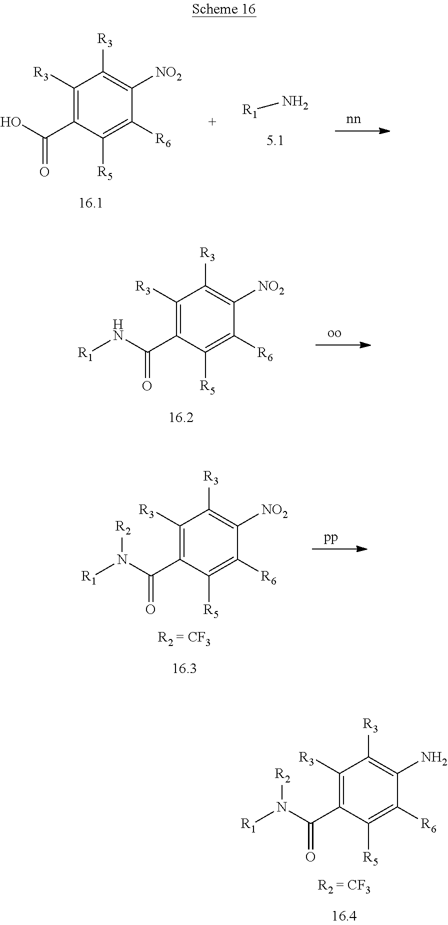

Alternatively, compounds of Formula 8.2, wherein R₁, R₂, R₃, R₄, R₅, R₆, R₇, R₈ and R₉ are as originally defined, can be prepared by the method shown in Scheme 17, steps qq. The compound of Formula 1.4, wherein R₃, R₄, R₅, and R₆ are as previously defined, can be treated with an amide, such as a compound of Formula 8.1, wherein R₇, R₈ and R₉ are as originally defined, in the presence of a dehydrating reagent, such as oxalyl chloride ((COCl)₂), in a solvent such as DCM, at a temperature from about 0° C. to about ambient temperature, followed by treatment with an amine, such as a compound of Formula 4.1, wherein R₁ and R₂ are as originally defined, in the presence of a base, such as triethylamine, at a temperature from about 0° C. to about ambient temperature, to afford compounds of Formula 8.2, wherein R₁, R₂, R₃, R₄, R₅, R₆, R₇, R₈ and R₉ are as originally defined, as shown in step qq.

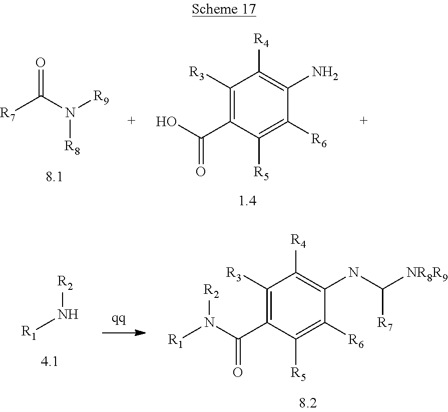

Compounds of Formula 18.1, wherein R₁, R₂, R₃, R₄, R₅, R₆, R₇, R₈ and R₉ are as originally defined, and X is S, can be prepared by the method shown in Scheme 18, step rr. The compound of Formula 8.2, wherein R₁, R₂, R₃, R₄, R₅, R₆, R₇, R₈ and R₉ are as originally defined, and X is O, can be treated with a thionation reagent, such as phosphorus pentasulfide (P₂S₅), in the presence of a dehydrating agent, such as 1,1,1,3,3,3-hexamethyldisiloxane, in a solvent, such as acetonitrile, at a temperature from about ambient temperature to reflux (˜81° C.), to afford compounds of Formula 18.1, wherein R₁, R₂, R₃, R₄, R₅, R₆, R₇, R₈ and R₉ are as originally defined, and X is S, as shown in step rr.

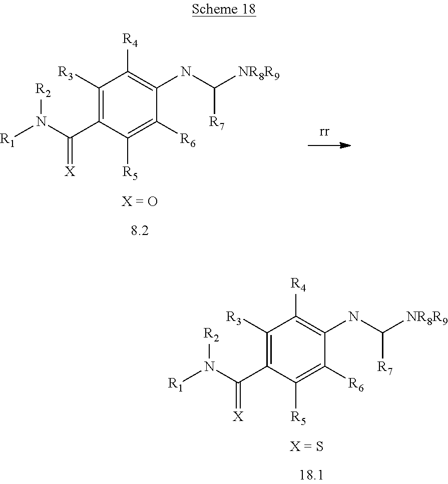

The following examples are for illustration purposes and are not to be construed as limiting this disclosure to only the embodiments disclosed in these examples.

Starting materials, reagents, and solvents that were obtained from commercial sources were used without further purification. Anhydrous solvents were purchased as Sure/Seal™ from Aldrich and were used as received. Melting points were obtained on a Thomas Hoover Unimelt capillary melting point apparatus or an OptiMelt Automated Melting Point System from Stanford Research Systems and are uncorrected. Examples using “room temperature” or “ambient temperature” were conducted in climate controlled laboratories with temperatures ranging from about 20° C. to about 24° C. Molecules are given their known names, named according to the naming program within ChemDraw (version 17.1.0.105 (19)). If such a program is unable to name a molecule, such molecule is named using conventional naming rules. ¹H NMR spectral data are in ppm (δ) and were recorded at 400, 500, or 600 MHz; ¹³C NMR spectral data are in ppm (δ) and were recorded at 101, 126, or 151 MHz, and ¹⁹F NMR spectral data are in ppm (δ) and were recorded at 376 or 471 MHz, unless otherwise stated.

### EXAMPLES

### Example 1A: Preparation of methyl 4-amino-5-iodo-2-methylbenzoate

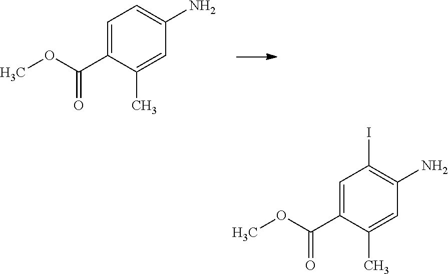

To a solution of methyl 4-amino-2-methylbenzoate (0.290 grams (g), 1.76 millimoles (mmol)) in DMF (1.5 milliliters (mL)) were added sodium periodate (0.140 g, 0.700 mmol) and iodine (I₂, 74.0 milligrams (mg), 1.41 mmol), respectively. The reaction mixture was stirred at 50° C. for 3 hours (h). The reaction mixture was diluted with a saturated sodium thiosulfate solution (5 mL). Solids were filtered and dried. The resulting product was triturated with ethyl acetate (EtOAc, 1 mL) and pentane (9 mL) to afford the title compound (0.22 g, 43% yield) as a pink solid: 1-14 NMR (400 MHz, CDCl₃) δ 8.27 (s, 1H), 6.54 (s, 1H), 4.38 (br s, 2H), 3.84 (s, 3H), 2.50 (s, 3H); ESIMS m/z 292 ([M+H]⁺).

### Example 1B: Preparation of methyl 4-acetamido-5-bromo-2-methoxybenzoate

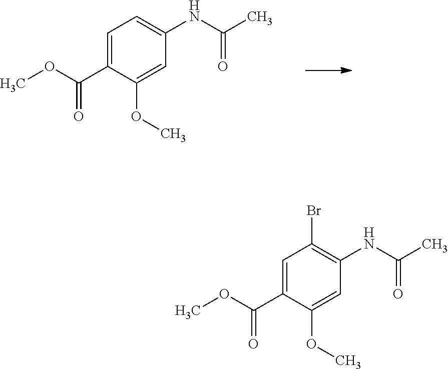 [SMILES: COC(=O)c1ccc(NC(C)=O)cc1OC ; COC(=O)c1cc(Br)c(NC(C)=O)cc1OC]

To a solution of methyl 4-acetamido-2-methoxybenzoate (4.04 g, 18.1 mmol) in DMF (80 mL) at 0° C. was added N-bromosuccinimide (3.22 g, 18.1 mmol). The mixture was stirred at 0° C. and allowed to warm slowly to room temperature while stirring overnight. The mixture was then diluted with water, and a precipitate formed. The precipitate was filtered and washed with additional water. The precipitate was dried under vacuum, providing an impure product, which was purified by flash column chromatography (silica gel (SiO₂), 0→100% ethyl acetate in hexane) to afford the title compound (3.89 g, 71% yield) as a white solid: ¹H NMR (400 MHz, CDCl₃) δ 8.32 (s, 1H), 8.04 (s, 1H), 7.76 (s, 1H), 3.93 (s, 3H), 3.87 (s, 3H), 2.28 (s, 3H); ¹³C NMR (101 MHz, CDCl₃) δ 166.28, 162.47, 157.58, 137.80, 132.74, 113.36, 102.13, 99.53, 54.06, 49.79, 22.92; ESIMS m/z 304 ([M+H]⁺).

### Example 1C: Preparation of 4-bromo-5-methyl-2-(trifluoromethyl)aniline

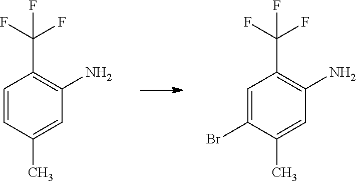

In a 25 mL vial, a solution of 5-methyl-2-(trifluoromethyl)aniline (1.00 g, 5.71 mmol) was prepared in DMF (18 mL). The reaction mixture was cooled to 0° C. in an ice-water bath. N-Bromosuccinimide (1.02 g, 5.71 mmol) was added in one portion. The reaction mixture was allowed to stir overnight, slowly warming to ambient temperature as the ice melted. After 18 h, the reaction was quenched with water (50 mL) and diluted with EtOAc (50 mL). The layers were separated, and the aqueous layer was extracted with EtOAc (3×50 mL). The combined organic layers were washed with brine (3×100 mL), dried over magnesium sulfate (MgSO₄), filtered, and concentrated to afford the title compound (1.31 g, 90% yield) as a dark yellow oil which was used without further purification: ¹H NMR (400 MHz, CDCl₃) δ 7.54 (s, 1H), 6.63 (s, 1H), 4.09 (s, 2H), 2.32 (s, 3H); ¹⁹F NMR (376 MHz, CDCl₃) δ-62.58; HRMS-ESI (m/z) [M+H]⁺ calcd for C₈H₈BrF₃N, 253.9787; found, 253.9778.

### Example 2A: Preparation of methyl 4-amino-2,5-dimethylbenzoate

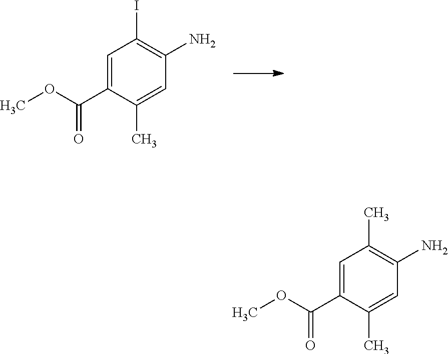

To a solution of methyl 4-amino-5-iodo-2-methylbenzoate (0.22 g, 0.75 mmol) in 1,4-dioxane (5 mL) was added cesium carbonate (0.980 g, 3.02 mmol), and the mixture was degassed for 5 minutes. [1,1′-Bis(diphenylphosphino)ferrocene]dichloropalladium(II) complex with dichloromethane (PdCl₂(dppf)DCM, 0.061 g, 0.07 mmol) and trimethylboroxine (0.23 g, 1.88 mmol) were then added, and the reaction mixture was heated to 120° C. for 1 h under microwave irradiation. The reaction mixture was diluted with water (15 mL) and extracted with EtOAc (2×40 mL). The combined organic layers were dried over anhydrous sodium sulfate (Na₂SO₄), filtered, and concentrated under reduced pressure. The resulting product was purified by flash column chromatography (SiO₂, 20→25% ethyl acetate in hexane) to afford the title compound (0.11 g, 84% yield) as a brown solid: ¹H NMR (500 MHz, CDCl₃) δ 7.71 (s, 1H), 6.45 (s, 1H), 3.93 (s, 2H), 3.82 (s, 3H), 2.51 (s, 3H), 2.12 (s, 3H); ¹³C NMR (126 MHz, CDCl₃) δ 167.88, 148.36, 140.61, 133.70, 118.62, 118.42, 117.02, 51.22, 21.87, 16.61; IR (thin film) 3373, 2947, 1690, 1624, 1560, 1434, 1258, 1155, 1064, 781 cm⁻¹; HRMS-ESI (m/z) [M+H]⁺ calcd for C₁₀H₁₄NO₂, 180.1019; found, 180.1021.

### Example 2B: Preparation of methyl 4-acetamido-2-methoxy-5-methylbenzoate

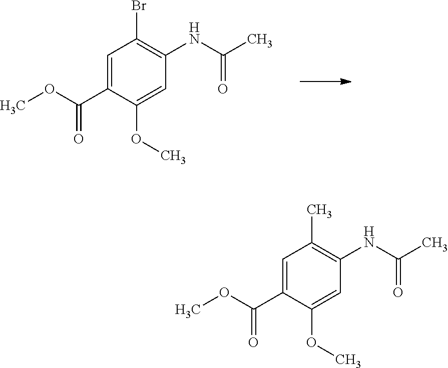 [SMILES: COC(=O)c1cc(Br)c(NC(C)=O)cc1OC ; COC(=O)c1cc(C)c(NC(C)=O)cc1OC]

Methyl 4-acetamido-5-bromo-2-methoxybenzoate (2.00 g, 6.62 mmol), methylboronic acid (0.594 g, 9.93 mmol), (2-dicyclohexylphosphino-2′,4′,6′-triisopropyl-1,1′-biphenyl)[2-(2′-amino-1,1′-biphenyl)]palladium(II) methanesulfonate (Xphos-Pd-G3, 0.112 g, 0.132 mmol), and potassium phosphate tribasic (2.81 g, 13.2 mmol) were dissolved/suspended in 1,4-dioxane (30.1 mL)/water (3.01 mL) and heated to 100° C. The mixture was stirred for 4 h at 100° C. The mixture was cooled to room temperature and diluted with DCM and water. The mixture was then passed through a phase separator, and the products extracted with DCM. The resulting product was purified by flash column chromatography (SiO₂, 0→100% ethyl acetate in hexane) to afford the title compound (658 mg, 42% yield) as a white solid and recovered starting material (866 mg, 43%): ¹H NMR (400 MHz, CDCl₃) δ 8.01 (s, 1H), 7.70-7.63 (m, 1H), 7.11 (s, 1H), 3.90 (s, 3H), 3.87 (s, 3H), 2.25 (s, 3H), 2.22 (s, 3H); ¹³C NMR (101 MHz, CDCl₃) δ 167.27, 165.10, 157.74, 139.71, 132.51, 131.72, 115.99, 103.58, 55.11, 50.80, 23.93, 15.45; ESIMS m/z 236 ([M−H]⁻).

### Example 3A: Preparation of 4-amino-2,5-dimethylbenzoic acid

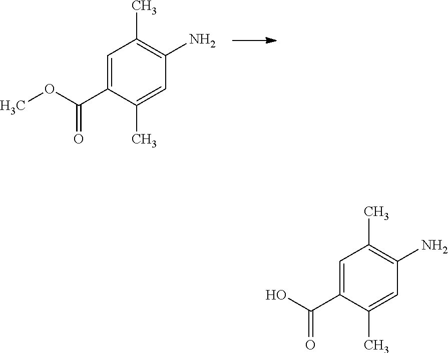

To a solution of methyl 4-amino-2,5-dimethylbenzoate (0.11 g, 0.69 mmol) in THF:MeOH:water (3:2:1, 2 mL) was added lithium hydroxide hydrate (0.073 g, 3.07 mmol), and the reaction mixture was stirred at 70° C. for 16 h. The reaction mixture was then acidified with acetic acid (0.5 mL). The precipitated solids were filtered and dried to afford the title compound (0.062 g, 54% yield) as a pale yellow solid: ¹H NMR (400 MHz, CDCl₃) δ 7.82 (s, 1H), 6.48 (s, 1H), 3.97 (br s, 2H), 2.54 (s, 3H), 2.14 (s, 3H) (no COOH); ESIMS m/z 166 ([M+H]⁺).

### Example 3B: Preparation of 4-amino-2-methoxy-5-methylbenzoic acid

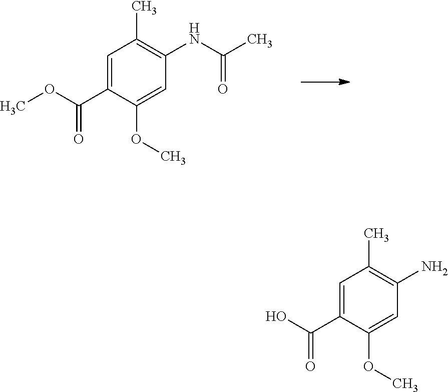 [SMILES: COC(=O)c1cc(C)c(NC(C)=O)cc1OC ; COc1cc(N)c(C)cc1C(=O)O]

In a 50 mL round-bottomed flask, methyl 4-acetamido-2-methoxy-5-methylbenzoate (0.658 g, 2.77 mmol) was dissolved/suspended in a 6 molar (M) aqueous potassium hydroxide (KOH solution). To the suspension at room temperature was added MeOH (5 mL). The mixture was then heated to 60° C. and stirred overnight. The reaction mixture was cooled to room temperature, diluted with water, and carefully acidified to pH ˜4-5 with 6 normal (N) hydrochloric acid (HCl) (dropwise). The products were extracted with EtOAc (3×). The combined organic layers were then dried with Na₂SO₄, filtered, and concentrated to afford the title compound (437 mg, 87% yield) as an off-white solid: ¹H NMR (500 MHz, CDCl₃) δ 7.84 (s, 1H), 6.25 (s, 1H), 4.19 (s, 3H), 3.98 (s, 3H), 2.11 (s, 3H); ¹³C NMR (126 MHz, CDCl₃) δ 165.97, 158.27, 151.07, 135.66, 115.42, 106.65, 96.62, 56.49, 16.15; ESIMS m/z 182 ([M+H]⁺).

### Example 4: Preparation of 1-bromo-5-chloro-2-methyl-4-nitrobenzene

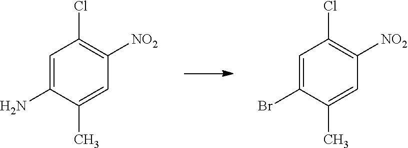

To a solution of 5-chloro-2-methyl-4-nitroaniline (5.30 g, 28.5 mmol) in acetic acid (53 mL) was added 47% aqueous HBr (7.7 mL) at room temperature. Sodium nitrite (NaNO₂, 1.96 g, 28.5 mmol) was then added over 45 minutes. The reaction mixture was stirred at 85° C. for 2 h. After 2 h, the reaction mixture was cooled to room temperature and poured into ice water (100 mL). The obtained solid was filtered, washed with water (100 mL), and dried to afford the title compound (5.50 g, 77% yield) as a pale yellow solid: ¹H NMR (400 MHz, CDCl₃) δ 7.79 (s, 1H), 7.52 (s, 1H), 2.45 (s, 3H).

### Example 5: Preparation of 4-bromo-2-chloro-5-methylaniline

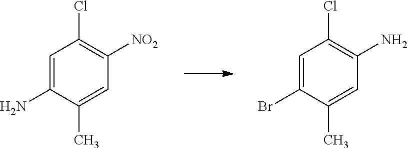

Iron powder (Fe °, 12.1 g, 221 mmol) and ammonium chloride (NH₄Cl, 11.7 g, 221 mmol) were added to a solution of 1-bromo-5-chloro-2-methyl-4-nitrobenzene (5.50 g, 22.1 mmol) in EtOH:water (55 mL, 1:1) at room temperature. The reaction mixture was stirred at 70° C. for 30 minutes. The reaction mixture was cooled to room temperature, and the solvent was concentrated under reduced pressure. The resulting material was diluted with water (30 mL) and filtered, and the solid was washed with EtOAc (30 mL). The aqueous layer was extracted with EtOAc (2×30 mL). The combined organic layers were dried over anhydrous Na₂SO₄, filtered, and concentrated under reduced pressure. The resultant product was purified via flash column chromatography (SiO₂, 3→5% ethyl acetate in petroleum ether) to afford the title compound (2.80 g, 58% yield) as an off-white solid: ¹H NMR (400 MHz, DMSO-d₆) δ 7.34 (s, 1H), 6.76 (s, 1H), 5.43 (br s, 2H), 2.19 (s, 3H); ESIMS m/z 220 ([M+H]⁺).

### Example 6A: Preparation of 4-amino-2,5-dichlorobenzonitrile

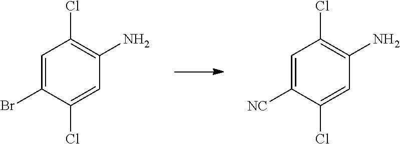 [SMILES: Nc1cc(Cl)c(Br)cc1Cl ; N#Cc1cc(Cl)c(N)cc1Cl]

To a solution of 4-bromo-2,5-dichloroaniline (2.00 g, 8.33 mmol) in N-methyl-2-pyrrolidone (NMP, 20 mL) was added copper(I) cyanide (CuCN, 2.20 g, 24.99 mmol), and the reaction mixture was heated to 180° C. for 1.5 h under microwave irradiation. The reaction mixture was poured into ice cold water (30 mL) and was extracted with EtOAc (3×60 mL). The organic layer was dried over anhydrous Na₂SO₄, filtered, and concentrated under reduced pressure to obtain the resulting product. The resulting product was purified by flash column chromatography (SiO₂, 15420% ethyl acetate in petroleum ether) to afford the title compound (1.00 g, 64% yield) as a pale yellow solid: ¹H NMR (400 MHz, CDCl₃) δ 7.83 (s, 1H), 6.92 (s, 1H), 6.73 (br s, 2H); ESIMS m/z 187 ([M+H]⁺).

### Example 6B: Preparation of 4-amino-2,5-dimethylbenzonitrile

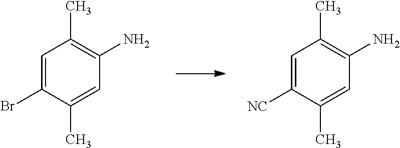

A solution of 4-bromo-2,5-dimethylaniline (15.0 g, 75.0 mmol) and zinc(II) cyanide (Zn(CN)₂, 9.60 g, 82.5 mmol) in DMF (150 mL) was degassed for 10 minutes. Tetrakis(triphenylphosphine)-palladium(0) (12.9 g, 11.3 mmol) was then added, and the reaction mixture was heated to 120° C. for 2 days in a sealed tube. After 2 days, the reaction mixture was poured into ice cold water (400 mL) and extracted with EtOAc (3×600 mL). The combined organic layers were dried over anhydrous Na₂SO₄, filtered, and concentrated under reduced pressure. The resulting product was purified by flash column chromatography (SiO₂, 15→20% ethyl acetate in petroleum ether) to afford the title compound (5.70 g, 52% yield) as pale yellow solid: ¹H NMR (400 MHz, CDCl₃) δ 7.25 (s, 1H), 6.50 (s, 1H), 3.98 (br s, 2H), 2.40 (s, 3H), 2.11 (s, 3H); ESIMS m/z 147 ([M+H]⁺).

### Example 6C: Preparation of methyl 4-amino-5-methoxy-2-methylbenzoate

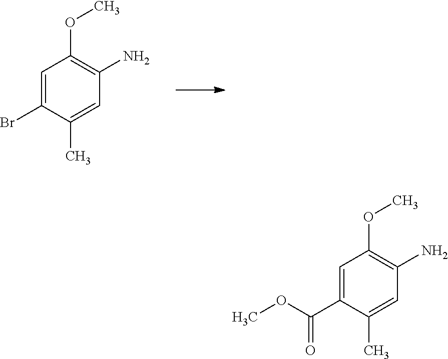

A solution of 4-bromo-2-methoxy-5-methylaniline (2.00 g, 9.30 mmol), palladium(II) acetate (0.302 g, 1.35 mmol), 1,4-bis(diphenylphosphino)butane (1.19 g, 2.79 mmol) and triethylamine (2.6 mL, 19 mmol) was prepared in MeOH (20 mL) in a 45 mL Parr reactor. The reactor was sealed and purged with carbon monoxide (CO, 3 cycles to 50-100 pounds per square inch (psi)). The reactor was then filled with CO to 400 psi, placed in a heating block, and heated to 130° C. for 24 h. The reaction mixture was concentrated, and the residue was dissolved in water (10 mL) and EtOAc (40 mL) and filtered through Celite®. The aqueous layer was extracted with EtOAc (3×20 mL). The combined organic layers were washed with brine (10 mL), dried over MgSO₄, filtered, and concentrated. The resulting product was purified by flash column chromatography (Sift, 0→40% ethyl acetate in petroleum ether) to afford the title compound (363 mg, 20% yield) as a rose red solid: ¹H NMR (400 MHz, CDCl₃) δ 7.42 (s, 1H), 6.50 (s, 1H), 4.12 (s, 2H), 3.87 (s, 3H), 3.84 (s, 3H), 2.49 (s, 3H); ESIMS m/z 196 ([M+H]⁺).

### Example 7A: Preparation of 4-amino-2,5-dichlorobenzoic acid

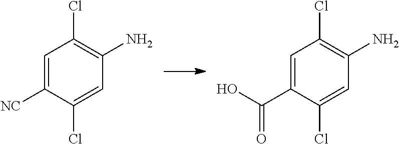 [SMILES: N#Cc1cc(Cl)c(N)cc1Cl ; Nc1cc(Cl)c(C(=O)O)cc1Cl]

To a solution of 4-amino-2,5-dichlorobenzonitrile (1.00 g, 5.37 mmol) in water (10 mL) was added KOH (6.00 g, 108 mmol) at room temperature, and the reaction mixture was heated to 120° C. for 2 days in a sealed tube. After 2 days, the reaction mixture was extracted with EtOAc (2×25 mL). The aqueous layer was acidified with acetic acid (12 mL) and was extracted with 10% MeOH in DCM (2×75 mL). The combined organic layers were dried over anhydrous Na₂SO₄, filtered, and concentrated under reduced pressure to afford the title compound (0.700 g, 63% yield) as a pale yellow solid which was used in the next step without further purification: ¹H NMR (400 MHz, CDCl₃) δ 7.61 (s, 1H), 6.77 (s, 1H), 5.89 (br s, 2H) (no COOH); ESIMS m/z 206 ([M+H]⁺).

### Example 7B: Preparation of 4-amino-5-methoxy-2-methylbenzoic acid

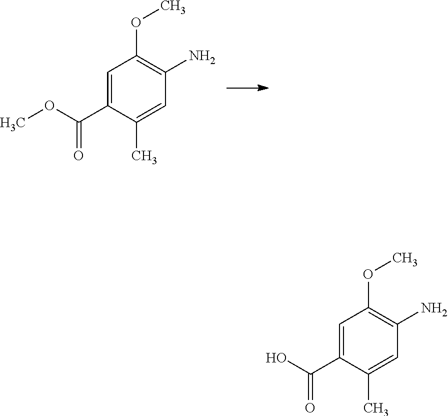

A solution of methyl 4-amino-5-methoxy-2-methylbenzoate (155 mg, 0.794 mmol) and lithium hydroxide (86 mg, 3.6 mmol) was prepared in 3:2:1 THF:MeOH:water (2.4 mL). The resulting dark purple reaction mixture was stirred at 70° C. overnight. 1 M HCl was then carefully added to acidify the reaction mixture to pH ˜4, and a solid precipitated. The aqueous layer was extracted with EtOAc (3×30 mL). The combined organic layers were dried over anhydrous MgSO₄, filtered, and concentrated under reduced pressure to afford the title compound (92 mg, 64% yield) as a dark green solid which was used in the next step without further purification: ¹H NMR (400 MHz, DMSO-d₆) δ 11.95 (s, 1H), 7.29 (s, 1H), 6.44 (s, 1H), 5.40 (s, 2H), 3.75 (s, 3H), 2.37 (s, 3H); ¹³C NMR (126 MHz, DMSO-d₆) δ 168.71, 143.69, 142.34, 134.93, 116.12, 115.89, 113.25, 21.98; IR (thin film) 3500, 3396, 2935, 2836, 1669, 1608, 1529, 1451, 1364, 1258, 1217, 1081, 1022, 867 cm⁻¹; HRMS-ESI (m/z) [M+H]⁺ calcd for C₉H₁₂NO₃, 182.0812; found, 182.0812.

### Example 8A: Preparation of 4-amino-N,2,5-trimethyl-N-phenethylbenzamide

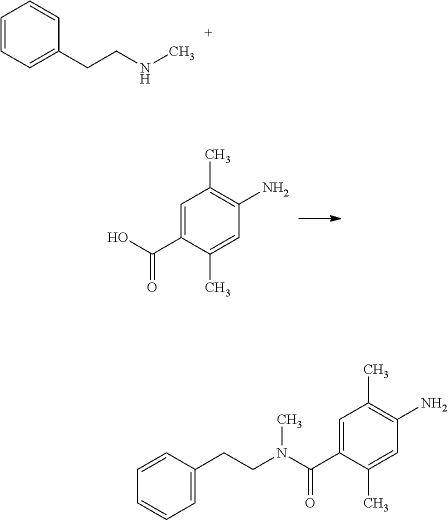

In a 20 mL vial equipped with a stir bar, 4-amino-2,5-dimethylbenzoic acid (150 mg, mmol), 1-ethyl-3-(3-dimethylaminopropyl)carbodiimide hydrochloride (EDCI, 261 mg, 1.36 mmol), N-methyl-2-phenylethan-1-amine (0.264 mL, 1.81 mmol), and 4-dimethylaminopyridine (DMAP, 11.1 mg, 0.091 mmol) were dissolved in DCM (5 mL). The vial was then capped, and the pale-yellow solution was stirred at room temperature overnight for 18 h. After 18 h, analysis by ultra performance liquid chromatography (UPLC) indicated consumption of starting material. The reaction mixture was diluted with DCM (3 mL) and washed with saturated sodium chloride (NaCl, brine) solution. The biphasic mixture was passed through a phase separator, and the organic layer was concentrated to a brown oil. The oil was purified by flash column chromatography (Sift, 0→40% 3:1 EtOAc:EtOH in hexanes) to afford the title compound (212 mg, 82% yield) as a yellow oil: ¹H NMR (500 MHz, CDCl₃) (rotamers present) δ 7.30 (m, 2H), 7.22 (m, 2H), 6.95 (app d, J=7.1 Hz, 1H), 6.75 (s, 0.5H), 6.43 (m, 1.5H), 3.77 (t, J=7.5 Hz, 1H), 3.64 (br s, 2H), 3.39 (t, J=7.4 Hz, 1H), 3.12 (s, 2H), 2.98 (t, J=7.6 Hz, 1H), 2.74 (m, 2H), 2.10 (m, 4H), 2.03 (m, 2H); ¹³C NMR (126 MHz, CDCl₃) (rotamers present) δ 172.48, 171.96, 144.96, 139.22, 138.29, 133.09, 132.93, 129.04, 128.94, 128.68, 128.60, 128.36, 127.32, 126.84, 126.61, 126.46, 119.61, 119.45, 116.32, 116.26, 52.52, 48.70, 37.18, 34.83, 33.65, 32.61, 18.72, 16.86; ESIMS m/z 283 ([M+H]⁺).

### Example 8B: Preparation of 4-amino-N-(2,6-dimethoxybenzyl)-2,5-dimethylbenzamide

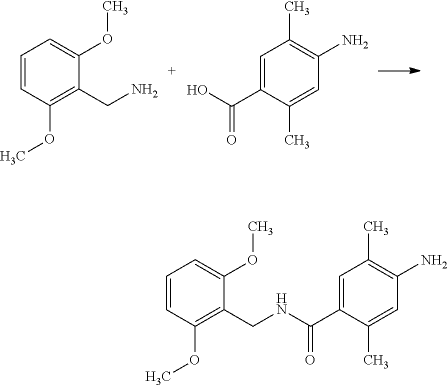 [SMILES: COc1cccc(OC)c1CN ; Cc1cc(C(=O)O)c(C)cc1N ; COc1cccc(OC)c1CNC(=O)c1cc(C)c(N)cc1C]

In a 20 mL vial equipped with a stir bar in open air, 4-amino-2,5-dimethylbenzoic acid (281 mg, 1.70 mmol), 1H-benzo[d][1,2,3]triazol-1-ol (276 mg, 2.04 mmol), and EDCI (392 mg, 2.04 mmol) were dissolved in DMF (10.0 mL). DIPEA (0.357 mL, 2.04 mmol) was then added. The vial was capped, and the dark orange solution was stirred for an hour at room temperature. After 1 h, (2,6-dimethoxyphenyl)methanamine (397 mg, 2.04 mmol) was added and the mixture was stirred overnight. The reaction was judged complete after 18 h as determined by UPLC analysis. The reaction mixture was diluted with EtOAc (15 mL) and was washed with deionized water (5×). The organic layer was washed with saturated aqueous sodium bicarbonate (NaHCO₃), dried over MgSO₄, filtered, and concentrated under reduced pressure to afford a dark yellow solid. The resulting material was purified by flash column chromatography (SiO₂, 0%→40% 3:1 EtOAc:EtOH in hexanes) to afford the title compound (270 mg, 51% yield) as an orange solid: ¹H NMR (500 MHz, CDCl₃) δ 7.23 (t, J=8.3 Hz, 1H), 7.10 (s, 1H), 6.57 (d, J=8.3 Hz, 2H), 6.45 (s, 1H), 6.08 (br s, 2H), 4.77-4.59 (m, 2H), 3.85 (s, 6H), 3.67 (s, 1H), 2.36 (s, 3H), 2.09 (s, 3H); ¹³C NMR (126 MHz, CDCl₃) δ 169.58, 158.87, 145.97, 135.69, 130.00, 129.05, 126.96, 119.02, 117.00, 114.51, 103.99, 56.00, 32.99, 19.99, 16.83; ESIMS m/z 315 ([M+H]⁺).

### Example 8C: Preparation of benzyl 4-amino-2,5-dimethylbenzoate

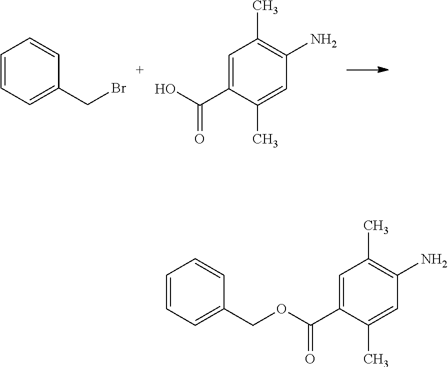 [SMILES: BrCc1ccccc1 ; Cc1cc(C(=O)O)c(C)cc1N ; Cc1cc(C(=O)OCc2ccccc2)c(C)cc1N]

In a 125 mL round-bottom flask, a solution of 4-amino-2,5-dimethylbenzoic acid (1.00 g, 6.05 mmol) was prepared in DMF (24.2 mL). Potassium carbonate (1.09 g, 7.87 mmol) and benzyl bromide (0.805 mL, 6.78 mmol) were added, and the resulting mixture was stirred at ambient temperature overnight. After 18 h, the reaction mixture was poured into ice cold water (100 mL) and extracted with DCM (3×100 mL). The combined organic layers were washed with brine (3×150 mL), passed through a phase separator, and concentrated to an oil. The resulting material was purified by flash column chromatography (Sift, 0%450% EtOAc in hexanes) to afford the title compound (1.15 g, 74% yield) as an off-white solid: ¹H NMR (400 MHz, CDCl₃) δ 7.76 (s, 1H), 7.46-7.40 (m, 2H), 7.40-7.27 (m, 3H), 6.45 (s, 1H), 5.29 (s, 2H), 3.90 (s, 2H), 2.52 (s, 3H), 2.11 (s, 3H); ¹³C NMR (101 MHz, CDCl₃) δ 167.10, 148.38, 140.85, 136.79, 133.82, 128.49, 128.06, 127.92, 118.64, 118.36, 117.04, 65.80, 22.02, 16.61; IR (thin film) 3375, 2927, 1690, 1622, 1561, 1252, 1150, 1052, 696 cm⁻¹; HRMS-ESI (m/z) [M+H]⁺ calcd for C₁₆H₁₈NO₂, 256.1332; found, 256.1338.

### Example 9: Preparation of 4-amino-N-(2,6-dimethoxybenzyl)-N,2,5-trimethylbenzamide

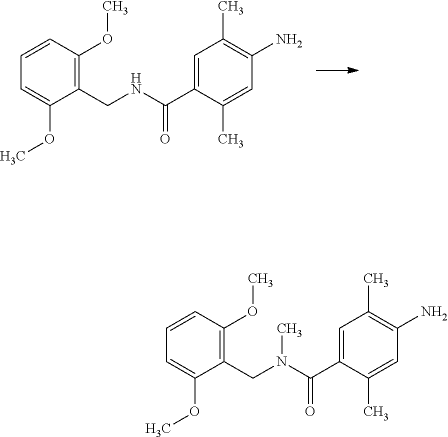 [SMILES: COc1cccc(OC)c1CNC(=O)c1cc(C)c(N)cc1C ; COc1cccc(OC)c1CN(C)C(=O)c1cc(C)c(N)cc1C]

To a vial equipped with a stir bar was added 4-amino-N-(2,6-dimethoxybenzyl)-2,5-dimethylbenzamide (205 mg, 0.652 mmol). The vial was evacuated and backfilled with nitrogen (3×) and fitted with a septum. THF (6.52 mL) was added, and the yellow solution was cooled to 0° C. Lithium bis(trimethylsilyl)amide (LHMDS, 1 M in THF, 0.720 mL, 0.717 mmol) was then added. The ice bath was removed, and the reaction mixture was allowed to warm to room temperature with constant stirring. After 1 h, the brown reaction mixture was cooled to 0° C., and iodomethane (MeI, 0.045 mL, 0.717 mmol) was added. The solution was allowed to warm to room temperature. After 2 h, the brown solution was diluted with DCM, and the reaction was quenched with water. The aqueous layer was extracted with DCM, and the organic extracts were washed with saturated aqueous NH₄Cl and saturated aqueous NaCl and passed through a phase separator. The solvent was removed to afford a brown solid. The solid material was purified via flash column chromatography (SiO₂, 0→40% 3:1 EtOAc:EtOH then 100% hexanes) to afford the title compound (111 mg, 48% yield) as an orange solid: mp 193-202° C.; ¹H NMR (500 MHz, CDCl₃) (rotamers observed) δ 7.20 (m, 1H), 7.04 (s, 0.6 H), 6.87 (s, 0.4H), 6.57 (d, J=8.4 Hz, 1H), 6.54-6.44 (m, 2H), 4.90 (br s, 1H), 4.47 (br s, 1H), 3.83 (s, 3H), 3.75 (s, 3H), 3.61 (s, 2H), 2.84 (s, 2H), 2.54 (s, 1H), 2.27 (s, 2H), 2.16 (m, 3H), 2.10 (s, 1H) (rotamers present); ESIMS m/z 329 ([M+H]⁺).

### Example 10A: Preparation of (E)-4-(((ethyl(methyl)amino)methylene)amino)-N2,5-trimethyl-N-phenethylbenzamide

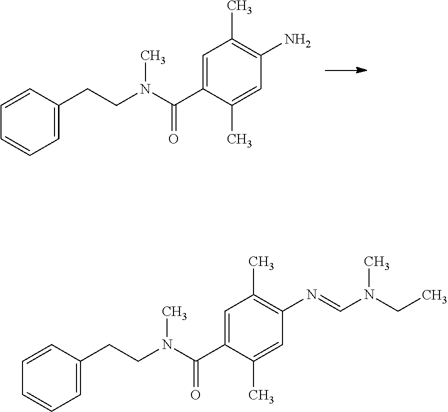 [SMILES: Cc1cc(C(=O)N(C)CCc2ccccc2)c(C)cc1N ; CCN(C)/C=N/c1cc(C)c(C(=O)N(C)CCc2ccccc2)cc1C]

In a 20 mL vial equipped with a stir bar were added 4-amino-N,2,5-trimethyl-N-phenethylbenzamide (195 mg, 0.691 mmol), trimethyl orthoformate (5 mL, 45.7 mmol), and p-toluenesulfonic acid monohydrate (13.1 mg, 0.069 mmol). The vial was fitted with an adaptor and Vigreux column. The yellow solution was heated to 105° C. and stirred for 16 h. After 16 h, UPLC analysis indicated consumption of starting material. The reaction mixture was diluted with DCM, and saturated aqueous NaHCO₃ was added. The biphasic mixture was passed through a phase separator, and the organic layer was concentrated. Note: Trimethyl orthoformate must be completely removed prior to the next step. The resulting residue was diluted with DCM (1 mL), and N-ethylmethylamine (0.119 mL, 1.38 mmol) was added. The reaction mixture was stirred for 18 h at room temperature. After 18 h, UPLC analysis indicated consumption of starting material, and the reaction mixture was concentrated to afford a brown oil. The resulting material was purified by flash column chromatography (C-18 reverse phase, 10→100% acetonitrile in water) to afford the title compound (154 mg, 63% yield) as a yellow oil: ¹H NMR (500 MHz, CDCl₃) (rotamers present) δ 7.39 (br s, 1H), 7.31 (m, 2H), 7.26-7.17 (m, 2H), 6.98-6.91 (m, 1H), 6.84 (s, 0.5H), 6.56 (s, 0.5H), 6.53 (d, J=4.6 Hz, 1H), 3.78 (t, J=7.5 Hz, 1H), 3.56-3.21 (m, 3H), 3.13 (s, 2H), 3.04-2.96 (m, 4H), 2.75 (t, J=7.4 Hz, 1H), 2.72 (s, 1H), 2.20 (s, 2H), 2.15 (s, 3H), 2.12 (s, 1H), 1.21 (td, J=7.1, 3.0 Hz, 3H); ¹³C NMR (151 MHz, CDCl₃) (rotamers present) δ 172.61, 172.14, 152.02, 151.48, 151.40, 139.27, 138.23, 132.14, 132.07, 130.86, 130.40, 129.07, 128.96, 128.86, 128.73, 128.63, 127.71, 127.63, 126.64, 126.49, 120.92, 120.87, 52.55, 48.71, 37.11, 34.92, 33.71, 32.59, 18.72, 18.64, 17.61, 17.59; IR (thin film) 3465, 3026, 2970, 2920, 2358, 2224, 1629, 1600, 1387, 1103, 700 cm⁻¹; HRMS-ESI (m/z) [M+H]⁺ calcd for C₂₂H₃₀N₃₀, 352.2383; found, 352.2398. Example 10B: Preparation of (E)-4-(((ethyl(methyl)amino)methylene)amino)-N,2,3-trimethyl-N-phenethylbenzamide.

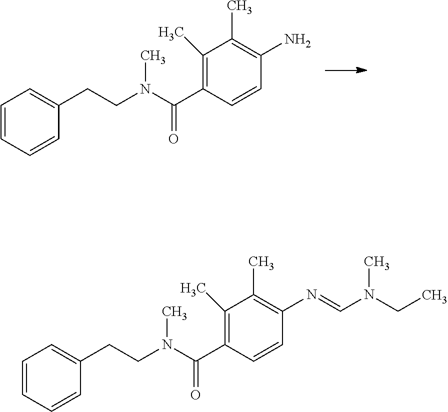 [SMILES: Cc1c(N)ccc(C(=O)N(C)CCc2ccccc2)c1C ; CCN(C)/C=N/c1ccc(C(=O)N(C)CCc2ccccc2)c(C)c1C]

In a 20 mL vial, a solution of N-ethyl-N-methylformamide (85% solution in toluene, 194 mg, 1.89 mmol) was prepared in DCM (1.5 mL). Oxalyl chloride (166 μL, 1.89 mmol) was added dropwide at room temperature. Immediate gas evolution was noted. This solution was allowed to stir at room temperature for 2 h. This solution was then added dropwise via syringe to a separate vial containing a solution of 4-amino-N,2,3-trimethyl-N-phenethylbenzamide (267 mg, 0.946 mmol) in DCM (5.0 mL). The reaction mixture was allowed to stir at room temperature for 18 h. The reaction was quenched by the dropwise addition of excess saturated aqueous sodium carbonate (Na₂CO₃). The biphasic mixture was passed through a phase separator, and the organic layer was concentrated. The residue was purified by flash column chromatography (SiO₂, 0% 100% EtOAc in hexanes) to give the title compound (260 mg, 78% yield) as a yellow oil: ¹H NMR (400 MHz, DMSO-d₆) δ 7.56 (d, J=33.9 Hz, 1H), 7.41-7.08 (m, 4H), 7.03-6.92 (m, 1H), 6.78-6.34 (m, 2H), 3.68 (s, 2H), 3.05-2.83 (m, 6H), 2.66 (s, 3H), 2.14 (d, J=3.7 Hz, 3H), 1.95 (dd, J=11.3, 9.5 Hz, 4H), 1.13 (td, J=7.1, 3.0 Hz, 3H); ESIMS m/z 352 ([M+H])⁺).

### Example 10C: Preparation of (E)-4-(((ethyl(methyl)amino)methylene)amino)-2,3-dimethyl-N-(4-methylbenzyl)benzamide

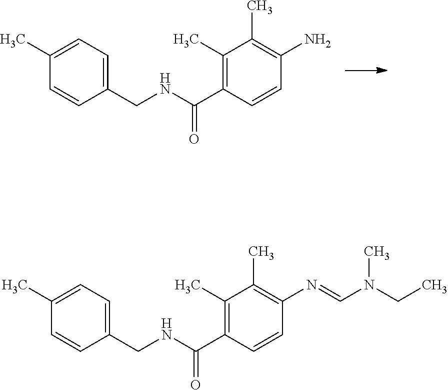 [SMILES: Cc1ccc(CNC(=O)c2ccc(N)c(C)c2C)cc1 ; CCN(C)/C=N/c1ccc(C(=O)NCc2ccc(C)cc2)c(C)c1C]

In a small vial equipped with a stir bar, a solution of 4-amino-2,3-dimethyl-N-(4-methylbenzyl)benzamide (66.0 mg, 0.246 mmol) was prepared in toluene (3 mL). To this solution was added N-(dimethoxymethyl)-N-methylethanamine (66.5 mg, 0.492 mmol). The reaction vessel was fitted with a reflux condenser, heated to 90° C., and stirred for 48 h. The reaction mixture was concentrated under a stream of nitrogen. The resulting material was purified via flash column chromatography (SiO₂, 0→90% EtOAc in hexane) to afford the title compound (83.0 mg, 99% yield) as a light yellow oil: ¹H NMR (400 MHz, CDCl₃) δ 7.36 (s, 1H), 7.26-7.23 (m, 2H), 7.16 (d, J=7.8 Hz, 2H), 7.11 (d, J=8.0 Hz, 1H), 6.57 (d, J=8.1 Hz, 1H), 5.93 (s, 1H), 4.58 (d, J=5.6 Hz, 2H), 3.52-3.25 (m, 2H), 3.00 (s, 3H), 2.36 (s, 3H), 2.34 (s, 3H), 2.23 (s, 3H), 1.21 (t, J=7.2 Hz, 3H); IR (thin film) 3345, 3233, 2971, 2930, 1715, 1591, 1519, 1328, 1252, 1142, 1037, 783 cm⁻¹; HRMS-ESI (m/z) [M+H]*calcd for C₂₁H₂₈N₃O, 338.2227; found, 338.2223.

### Example 10D: Preparation of benzyl (E)-4-(((ethyl(methyl)amino)methylene)amino)-2,5-dimethylbenzoate

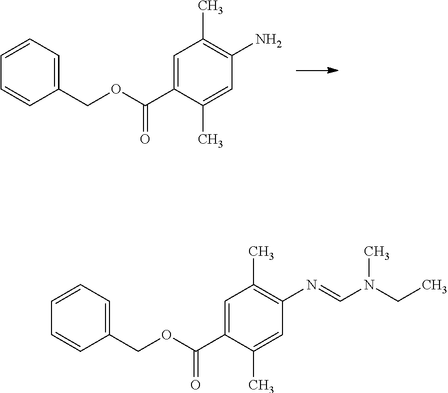 [SMILES: Cc1cc(C(=O)OCc2ccccc2)c(C)cc1N ; CCN(C)/C=N/c1cc(C)c(C(=O)OCc2ccccc2)cc1C]

In a 40 mL vial, a solution of benzyl 4-amino-2,5-dimethylbenzoate (1.15 g, 4.50 mmol) was prepared in trimethyl orthoformate (14.8 mL, 135 mmol). p-Toluenesulfonic acid monohydrate (0.0860 g, 0.450 mmol) was added, and the reaction mixture was stirred at reflux for 3 h. After 3 h, the reaction mixture was concentrated to a pale yellow oil. The residue was dissolved in DCM (4.50 mL), and N-ethylmethylamine (0.619 mL, 7.21 mmol) was added dropwise via syringe. The solution was heated to 40° C. and stirred for 3 h. After 3 h, the resulting material was purified by flash column chromatography (SiO₂, 0%→100% EtOAc in hexanes) to give the title compound (1.35 g, 92% yield) as an orange oil: ¹H NMR (400 MHz, CDCl₃) δ 7.80 (s, 1H), 7.51-7.42 (m, 2H), 7.42-7.29 (m, 4H), 6.57 (s, 1H), 5.31 (s, 2H), 3.41 (d, J=71.5 Hz, 2H), 3.01 (s, 3H), 2.55 (s, 3H), 2.23 (s, 3H), 1.21 (t, J=7.1 Hz, 3H); ESIMS m/z 325 ([M+H]⁺).

### Example 10E: Preparation of (E)-N′-(4-bromo-2-methyl-5-(trifluoromethyl)phenyl)-N-ethyl-N-methylformimidamide

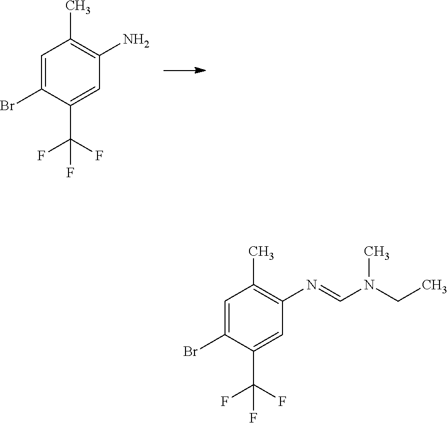 [SMILES: Cc1cc(Br)c(C(F)(F)F)cc1N ; CCN(C)/C=N/c1cc(C(F)(F)F)c(Br)cc1C]

Preparation of Vilsmeier reagent: In a 40 mL vial equipped with a stir bar, a solution of N-ethyl-N-methylformamide (0.538 g, 6.17 mmol) was prepared in dry DCM (7 mL). The solution was cooled to 0° C., and oxalyl chloride (0.529 mL, 6.17 mmol) was added dropwise. The vial was removed from the ice bath and allowed to warm to room temperature while stirring for 30 minutes. Reaction with substrate: In a separate 40 mL vial, a solution of 4-bromo-2-methyl-5-(trifluoromethyl)aniline (1.25 g, 4.94 mmol) was prepared in dry DCM (5 mL). To the resulting solution, the Vilsmeier reagent prepared above was added dropwise, and the reaction mixture was stirred at room temperature for 1.5 h. Saturated aqueous Na₂CO₃ was added until the pH was basic, and then water was added. The organic phase was separated with a phase separator, and the material was concentrated. The resulting material was purified by flash column chromatography (SiO₂, 0%→10% EtOAc in hexanes) to give the title compound (1.41 g, 97% yield) as an orange oil: ¹H NMR (500 MHz, CDCl₃) δ 7.42 (s, 2H), 7.01 (s, 1H), 3.55-3.24 (m, 2H), 3.01 (s, 3H), 2.26 (s, 3H), 1.22 (t, J=7.4 Hz, 3H); ¹⁹F NMR (471 MHz, CDCl₃) δ-61.97; ESIMS m z 324 ([M+H]⁺).

### Example 10F: Preparation of (E)-4-(((ethyl(methyl)amino)methylene)amino)-2,5-dimethylbenzoic acid hydrochloride

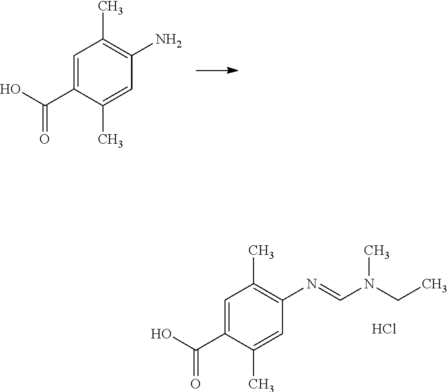 [SMILES: Cc1cc(C(=O)O)c(C)cc1N ; CCN(C)/C=N/c1cc(C)c(C(=O)O)cc1C ; Cl]

Preparation of Vilsmeier reagent: Under air, a 40-mL vial equipped with a magnetic stir bar was charged with N-ethyl-N-methylformamide (2.07 g, 23.2 mmol) and DCM (20.0 mL). The solution was placed at 0° C., and oxalyl chloride (1.97 mL, 23.2 mmol) was added dropwise. The vial was removed from the ice bath and stirred at room temperature for 1 h. Reaction with substrate: Under air, a separate 100-mL flask was charged with 4-amino-2,5-dimethylbenzoic acid (1.54 g, 9.30 mmol) and DCM (15.0 mL). To the resulting solution, the Vilsmeier reagent prepared above was added dropwise, and the reaction mixture was stirred for 1.5 h. Water (0.837 mL, 46.5 mmol) was then added, and the resulting solution was stirred for 30 minutes. MeOH (10 mL) was added to the solution and the solvent was removed under reduced pressure. Acetone was added and the formed solid was filtered and washed with portions of acetone and ethyl acetate to afford the title compound (2.44 g, 97% yield) as a white solid: ¹H NMR (500 MHz, DMSO-d₆) δ 12.96 (s, 1H), 11.67-11.21 (m, 1H), 8.58-8.33 (m, 1H), 7.76 (s, 1H), 7.33 (d, J=13.9 Hz, 1H), 3.89-3.58 (m, 2H), 2.54-2.32 (m, 9H), 1.26 (dt, J=12.8, 6.9 Hz, 3H); ESIMS m z 235 ([M−Cl]⁺).

### Example 11: Preparation of (E)-4-(((ethyl(methyl)amino)methylene)amino)-2,5-dimethylbenzoic acid

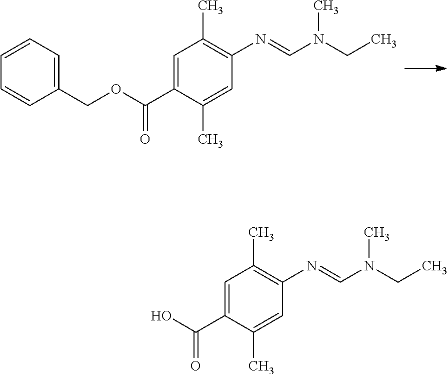 [SMILES: CCN(C)/C=N/c1cc(C)c(C(=O)OCc2ccccc2)cc1C ; CCN(C)/C=N/c1cc(C)c(C(=O)O)cc1C]

In a 125 mL round-bottom flask, a solution of benzyl (E)-4-(((ethyl(methyl)amino)methylene)amino)-2,5-dimethylbenzoate (1.15 g, 3.55 mmol) was prepared in EtOAc (11.9 mL) and cyclohexene (5.92 mL). To this solution was added 5% palladium on carbon (0.378 g, 0.178 mmol). The flask was fitted with a reflux condenser, and the reaction mixture was stirred at 70° C. for 6 h. After 6 h, the reaction mixture was filtered through a plug of Celite*, which was washed with EtOAc. The filtrate was concentrated to an oil. The resulting material was purified by flash column chromatography (SiO₂, 0%→100% acetone in hexanes) to give the title compound (736 mg, 88% yield) as an off-white solid: mp 122-125° C.; ¹H NMR (500 MHz, DMSO-d₆) δ 12.18 (s, 1H), 7.75 (s, 1H), 7.63 (s, 1H), 6.66 (d, J=9.5 Hz, 1H), 3.50-3.28 (m, 2H), 2.97 (d, J=31.8 Hz, 3H), 2.45 (s, 3H), 2.15 (s, 3H), 1.13 (d, J=7.2 Hz, 3H); ¹³C NMR (126 MHz, DMSO-d₆) δ 168.50, 153.96, 152.61, 138.40, 132.41, 127.73, 122.30, 121.19, 46.86, 31.48, 21.48, 17.29, 14.09; ESIMS m/z 235 ([M+H]⁺).

### Example 12A: Preparation of (E)-4-(((ethyl(methyl)amino)methylene)amino)-2,5-dimethyl-N-phenylbenzamide

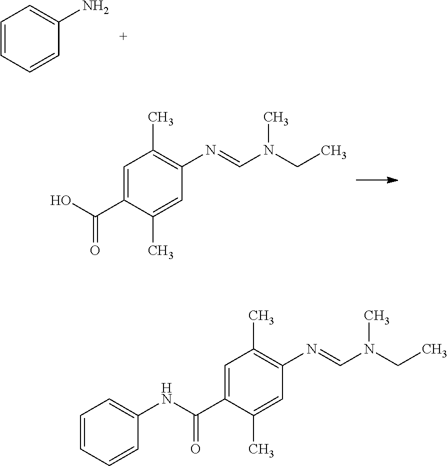 [SMILES: Nc1ccccc1 ; CCN(C)/C=N/c1cc(C)c(C(=O)O)cc1C ; CCN(C)/C=N/c1cc(C)c(C(=O)Nc2ccccc2)cc1C]

In a 25 mL vial, a solution of (E)-4-(((ethyl(methyl)amino)methylene)amino)-2,5-dimethylbenzoic acid (40.0 mg, 0.171 mmol), aniline (17.0 mg, 0.179 mmol), DMAP (25.0 mg, 0.205 mmol) and 1H-benzo[d][1,2,3]triazol-1-ol (29.0 mg, 0.188 mmol) was prepared in DMF (0.569 mL). EDCI (36.0 mg, 0.188 mmol) was added at room temperature, and the reaction mixture was stirred overnight. The reaction mixture was poured into deionized water and extracted with EtOAc (3×). The combined organic layers were concentrated to dryness. The resulting material was purified via flash column chromatography (SiO₂, 0→30% EtOAc in hexane) to afford the title compound (16.0 mg, 30% yield) as a white solid: mp 147-149° C.; ¹H NMR (400 MHz, CDCl₃) δ 7.61 (d, J=8.0 Hz, 2H), 7.45 (s, 2H), 7.36 (t, J=7.9 Hz, 2H), 7.31 (s, 1H), 7.16-7.08 (m, 1H), 6.62 (s, 1H), 3.35 (br s, 2H), 3.02 (s, 3H), 2.47 (s, 3H), 2.26 (s, 3H), 1.23 (t, J=7.1 Hz, 3H); ESIMS m/z 311 ([M+2H]⁺).

### Example 12B: Preparation of (E)-N-(2,6-difluorobenzyl)-4-(((ethyl(methyl)amino)methylene)amino)-N,2,5-trimethylbenzamide

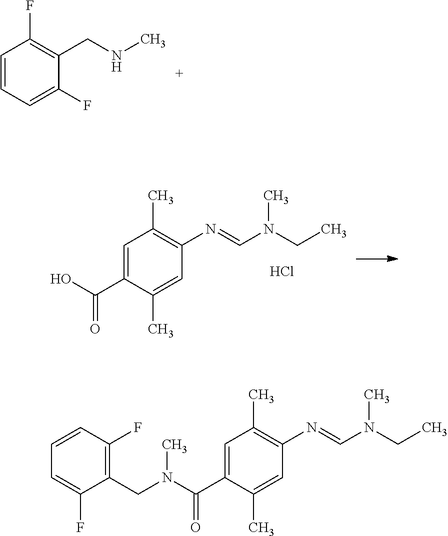 [SMILES: CNCc1c(F)cccc1F ; CCN(C)/C=N/c1cc(C)c(C(=O)O)cc1C ; Cl ; CCN(C)/C=N/c1cc(C)c(C(=O)N(C)Cc2c(F)cccc2F)cc1C]

A 20 mL vial was equipped with a magnetic stir bar and (E)-4-(((ethyl(methyl)amino)methylene)amino)-2,5-dimethylbenzoic acid hydrochloride (150 mg, 0.554 mmol), 1-(2,6-difluorophenyl)-N-methylmethanamine (131 mg, 0.831 mmol), N,N-dimethyl-3-(((methylimino)methylene)amino)propan-1-amine hydrochloride (148 mg, 0.831 mmol), and DMAP (6.77 mg, 0.0550 mmol) were added, followed by dry DCM (5.00 mL). Triethylamine (0.193 mL, 1.39 mmol) was then added, and the solution was stirred at room temperature overnight. Brine (7 mL) and DCM (7 mL) were then added to the reaction mixture, and the material was passed through a phase separator. The organic phase was concentrated under reduced pressure. The resultant product was purified by flash column chromatography (SiO₂, 0→4% MeOH in DCM) to afford the title product (207 mg, quantitative) as a brown oil: ¹H NMR (400 MHz, CDCl₃) δ 7.50 (td, J=8.5, 6.4 Hz, 1H), 7.41 (s, 1H), 6.99-6.73 (m, 3H), 6.57 (d, J=5.7 Hz, 1H), 3.33 (s, 3H), 3.01 (d, J=14.4 Hz, 4H), 2.77 (s, 2H), 2.26-2.16 (m, 6H), 1.88 (s, 1H), 1.21 (t, J=7.1 Hz, 3H); ESIMS m/z 374 ([M+H]⁺).

### Example 12C: Preparation of (E)-N′-(4-(1H-imidazole-1-carbonyl)-2,5-dimethylphenyl)-N-ethyl-N-methylformimidamide

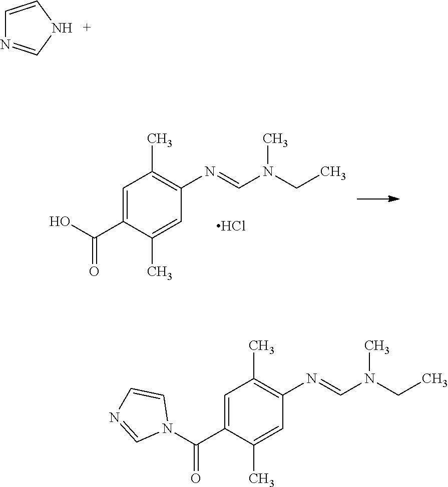

In a 25 mL vial, a solution of (E)-4-(((ethyl(methyl)amino)methylene)amino)-2,5-dimethylbenzoic acid hydrochloride (0.329 g, 1.22 mmol), 1H-imidazole (0.165 g, 2.43 mmol), EDCI (0.419 g, 2.187 mmol), and DMAP (0.0450 g, 0.365 mmol was prepared in DCM (2.43 mL). Triethylamine (0.847 mL, 6.08 mmol) was added at room temperature, and the solution was stirred overnight. After 16 h, the reaction was quenched with saturated aqueous NaHCO₃ (20 mL) and diluted with DCM (10 mL). The reaction mixture was passed through a phase separator and concentrated. The resulting material was purified by flash column chromatography (SiO₂, 0%→4% methanol in DCM) to give the title compound (261 mg, 76% yield) as an off-white solid: ¹H NMR (500 MHz, CDCl₃) δ 7.93 (t, J=1.1 Hz, 1H), 7.53 (s, 1H), 7.49 (t, J=1.5 Hz, 1H), 7.21 (s, 1H), 7.12 (dd, J=1.6, 0.8 Hz, 1H), 6.67 (s, 1H), 3.68-3.22 (m, 2H), 3.04 (s, 3H), 2.58, 2.37 (s, 3H), 2.24 (s, 3H), 1.29-1.21 (m, 3H); ¹³C NMR (126 MHz, CDCl₃) δ 166.80, 154.87, 152.06, 138.53, 137.71, 131.37, 130.73, 129.26, 124.81, 121.80, 117.88, 48.09, 32.19, 19.73, 17.65, 14.49; IR (thin film) 3153, 1976, 2926, 2253, 1707, 1634, 1596, 1365, 1085, 906, 729 cm⁻¹; HRMS-ESI (m z) [M+H]⁺ calcd for C₁₆H₂₁N₄O, 284.1637; found, 284.1636.

### Example 13: Preparation of (E)-4-(((ethyl(methyl)amino)methylene)amino)-5-methyl-2-(trifluoromethyl)benzoic acid hydrochloride

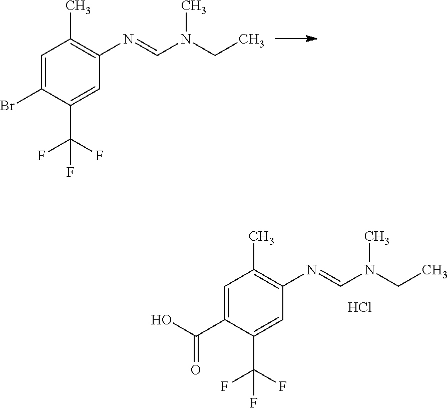 [SMILES: CCN(C)/C=N/c1cc(C(F)(F)F)c(Br)cc1C ; CCN(C)/C=N/c1cc(C(F)(F)F)c(C(=O)O)cc1C ; Cl]

A flame-dried Schlenk flask equipped with a magnetic stir bar and under a nitrogen atmosphere was charged with (E)-N′-(4-bromo-2-methyl-5-(trifluoromethyl)phenyl)-N-ethyl-N-methylformimidamide (1.34 g, 4.16 mmol) and dry THF (12.0 mL). The resulting solution was cooled to −78° C.; n-butyllithium (1.80 mL, 4.60 mmol, 2.5 M in hexanes) was added dropwise, and the reaction mixture was stirred for 30 minutes at −78° C. A second flame-dried Schlenk flask under nitrogen was charged with dry THF (8.00 mL) and cooled to −78° C. Crushed dry ice (˜10 g) was added with a funnel, and the solution was stirred for 10 seconds. The suspended dry ice was filtered under a nitrogen stream and quickly added to the Schlenk flask containing the butyllithium solution. The Schlenk flask was left open under a nitrogen stream, and the solution was stirred for 15 minutes at −78° C. The solution was allowed to warm to ambient temperature, and MeOH (10 mL) and HCl (2.6 mL, 4.0 M in 1,4-dioxane) were added. The solvent was removed under reduced pressure, and acetone was added to the resulting material to induce the formation of a white precipitate, which was collected by filtration and washed with small portions of acetone to afford the title compound (1.24 g, 92% yield) as a white solid: ¹H NMR (500 MHz, DMSO-d₆) δ 13.68 (s, 1H), 11.79-11.32 (m, 1H), 8.72-8.28 (m, 1H), 7.91 (d, J=6.2 Hz, 1H), 7.80 (s, 1H), 3.88-3.62 (m, 2H), 3.34-3.31 (m, 3H), 2.49 (s, 3H), 1.27 (dt, J=10.7, 7.1 Hz, 3H); ¹⁹F NMR (471 MHz, DMSO-d₆) δ-57.75, -57.76; ESIMS m z 289 ([M−Cl])⁺).

### Example 14: Preparation of (E)-N′-(2,5-dimethyl-4-(5-(trifluoromethyl)-1H-indole-1-carbonyl)phenyl)-N-ethyl-N-methylformimidamide

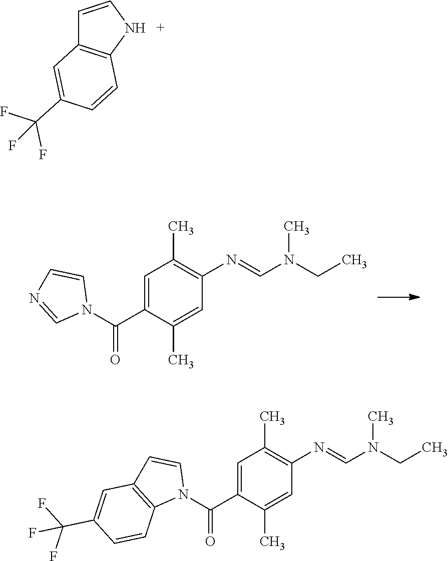

To a small vial equipped with a stir bar was added 5-(trifluoromethyl)-1H-indole (75.0 mg, 0.405 mmol) and (E)-N-(4-(1H-imidazole-1-carbonyl)-2,5-dimethylphenyl)-N-ethyl-N-methylformimidamide (134 mg, 0.469 mmol). The vial was evacuated and backfilled with nitrogen (3×) and capped with a screw cap. The solids were dissolved in anhydrous acetonitrile (4.05 mL), and 1,8-diazabicyclo[5.4.0]undec-7-ene (DBU, 12.2 μL, 0.0810 mmol) was added. The reaction mixture was allowed to stir at room temperature overnight. UPLC analysis indicated consumption of starting material. The reaction was quenched by the addition of brine (3 mL), and the reaction mixture was extracted with EtOAc (3×5 mL). The organic extracts were concentrated to afford a yellow oil. The resulting oil was purified via flash column chromatography (Cis reverse phase, 0→100% acetonitrile in water) to afford the title compound (113 mg, 70% yield) as a pale yellow solid: mp 89-92° C.; ¹H NMR (500 MHz, CDCl₃) δ 8.35 (d, J=8.7 Hz, 1H), 7.89-7.85 (m, 1H), 7.57 (dd, J=8.8, 1.8 Hz, 1H), 7.53 (s, 1H), 7.31 (d, J=3.8 Hz, 1H), 7.18 (s, 1H), 6.68 (s, 1H), 6.63 (dd, J=3.8, 0.8 Hz, 1H), 3.63-3.27 (m, 2H), 3.05 (s, 3H), 2.28 (s, 3H), 2.26 (s, 3H), 1.25 (t, J=7.1 Hz, 3H); ¹⁹F NMR (471 MHz, CDCl₃) δ-61.13; IR (thin film) 3735, 3628, 3119, 2974, 2923, 2359, 2342, 1690, 1633, 1594, 1442, 1323, 1256, 1057, 893, 733 cm⁻¹; HRMS-ESI (m/z) [M+H]⁺ calcd for C₂₂H₂₃F₃N₃O, 402.1788; found, 402.1786.

### Example 15: Preparation of (E)-4-(((ethyl(methyl)amino)methylene)amino)-N,2,5-trimethyl-N-(4-methylbenzyl)benzamide

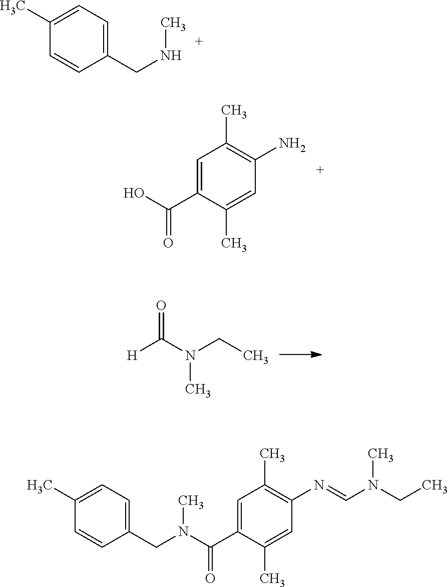

To a solution of N-ethyl-N-methylformamide (218 mg, 2.50 mmol) in dry DCM (4.00 mL) was added oxalyl chloride (212 μl, 2.50 mmol) dropwise at 0° C., and the reaction mixture was stirred at ambient temperature for 30 minutes. The resulting solution was added dropwise to a separate solution of 4-amino-2,5-dimethylbenzoic acid (165 mg, 1.00 mmol) in dry DCM (3.00 mL), and the reaction mixture was stirred at ambient temperature for 1 h. The solution was then cooled to 0° C. in an ice water bath, and a solution of triethylamine (0.700 mL, 5.00 mmol) in DCM (3.00 mL) was added dropwise, and the mixture was stirred for 30 minutes at 0° C. A solution of N-methyl-1-(p-tolyl)methanamine (270 mg, 2.00 mmol) in DCM (2.00 mL) was added dropwise, and the reaction mixture was stirred at ambient temperature for 1.5 h. Brine (5 mL) was added, and the organic phase was separated and concentrated under reduced pressure. The resulting material was purified by flash column chromatography (SiO₂, 0→5% MeOH in DCM) to afford the title compound (257 mg, 73% yield) as a brown oil: ¹H NMR (400 MHz, DMSO-d₆) δ 7.62 (d, J=33.4 Hz, 1H), 7.24 (d, J=7.8 Hz, 1H), 7.21-7.11 (m, 2H), 7.01 (d, J=7.7 Hz, 1H), 6.90 (d, J=24.6 Hz, 1H), 6.63 (s, 1H), 4.61 (s, 1H), 4.31 (s, 1H), 3.49-3.26 (m, 2H), 3.00-2.87 (m, 3H), 2.85 (s, 1H), 2.64 (s, 2H), 2.28 (d, J=12.2 Hz, 3H), 2.23-2.05 (m, 6H), 1.20-1.02 (m, 3H); IR (think film) 2918, 1626, 1598, 1385, 1263, 1101, 1085, 729 cm⁻¹; HRMS-ESI (m/z) [M+H]⁺ calcd for C₂₂H₃₀N₃O, 352.2383; found, 352.2398.

### Example 16: Preparation of (E)-4-(((ethyl(methyl)amino)methylene)amino)-N,2,5-trimethyl-N-(3-(trifluoromethyl)benzyl)benzothioamide

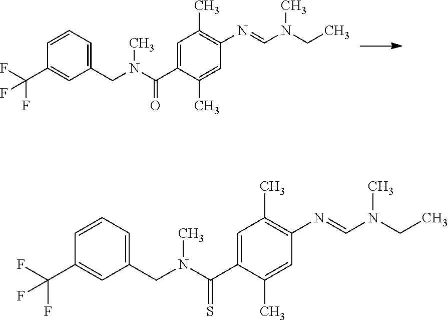 [SMILES: CCN(C)/C=N/c1cc(C)c(C(=O)N(C)Cc2cccc(C(F)(F)F)c2)cc1C ; CCN(C)/C=N/c1cc(C)c(C(=S)N(C)Cc2cccc(C(F)(F)F)c2)cc1C]

To a 25 mL vial equipped with a stir bar were added (E)-4-(((ethyl(methyl)amino)methylene)amino)-N,2,5-trimethyl-N-(3-(trifluoromethyl)benzyl)benzamide (50.0 mg, 0.123 mmol) and anhydrous acetonitrile (1.50 mL). 1,1,1,3,3,3-Hexamethyldisiloxane (138 μL, 0.617 mmol) and phosphorus pentasulfide (54.8 mg, 0.247 mmol) were added. The reaction mixture was heated to 80° C. with constant stirring overnight. UPLC indicated product formation. The reaction mixture was diluted with DCM (3 mL), and the reaction was quenched with aqueous 1 M NaOH (3 mL). The mixture was passed through a phase separator and concentrated to a yellow oil. The resulting oil was purified via flash column chromatography (SiO₂, 0→100% 3:1 ethyl acetate:ethanol in hexanes) to afford the title compound (41.0 mg, 79% yield) as a yellow oil: Rotamers observed in the NMR spectra. ¹H NMR (500 MHz, CDCl₃) δ 7.70 (d, J=6.9 Hz, 1H), 7.62-7.43 (m, 2H), 7.40 (s, 1H), 7.36-7.27 (m, 1H), 6.99-6.88 (m, 1H), 6.53 (s, 1H), 5.83-4.95 (m, 1H), 4.93-4.33 (m, 1H), 3.49 (s, 1H), 3.33 (s, 2H), 2.99 (s, 2H), 2.98 (s, 1H), 2.93 (s, 2H), 2.24-2.14 (m, 6H), 1.20 (td, J=7.1, 5.0 Hz, 3H); ¹⁹F NMR (471 MHz, CDCl₃) δ-62.65, -62.78; IR (thin film) 3031, 3170, 2972, 2921, 2138, 1632, 1598, 1493, 1328, 1164, 1124, 1074, 974, 703 cm⁻¹; HRMS-ESI (m/z) [M+H]⁺ calcd for C₂₂H₂₇F₃N₃S, 422.1833; found, 422.1888.

### Example 17A: Preparation of 2,2,2-trifluoro-N-(3-(trifluoromethyl)benzyl)ethan-1-amine

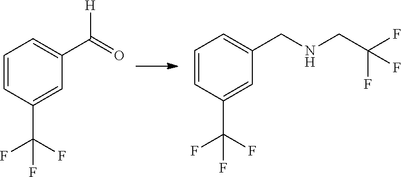 [SMILES: O=Cc1cccc(C(F)(F)F)c1 ; FC(F)(F)CNCc1cccc(C(F)(F)F)c1]

To a small vial equipped with a stir bar were added 3-(trifluoromethyl)benzaldehyde (0.535 mL, 4.00 mmol), 2,2,2-trifluoroethan-1-amine (0.377 mL, 4.80 mmol), and then anhydrous MeOH (5.33 mL). The reaction mixture was cooled to 0° C. in an ice/water bath. Sodium cyanoborohydride (452 mg, 7.20 mmol) was added in portions, followed by the addition of acetic acid (0.286 mL, 5.00 mmol). The reaction mixture was allowed to stir at room temperature for 18 h. After 18 h, UPLC indicated consumption of starting material. MeOH was removed from the resulting yellow solution under reduced pressure. Water and diethyl ether were added to the resulting oil, and the biphasic mixture was extracted with diethyl ether. The organic layers were combined, dried over Na₂SO₄, filtered, and concentrated under reduced pressure to yield the title compound (924 mg, 90% yield) as a colorless oil that was used in the next step without further purification: ¹H NMR (500 MHz, CDCl₃) δ 7.62 (s, 1H), 7.54 (d, J=8.5 Hz, 2H), 7.46 (t, J=7.7 Hz, 1H), 3.98 (s, 2H), 3.21 (q, J=9.4 Hz, 2H), 2.38 (br s, 1H); ¹⁹F NMR (471 MHz, CDCl₃) δ-62.64, -71.43 (t, J=9.6 Hz); ESIMS m z 258 ([M+H]⁺).

### Example 17B: Preparation of 1-(2,6-difluorophenyl)-N-methylmethanamine

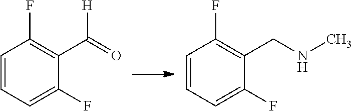

A 20-mL vial equipped with a magnetic stir bar was charged with 2,6-difluorobenzaldehyde (0.231 mL, 2.11 mmol) and dry MeOH (8.12 mL). Methanamine (2.11 mL, 4.22 mmol) was added, and the solution was stirred for 3 h at room temperature. Sodium borohydride (0.120 g, 3.17 mmol) was added portionwise, and the resulting solution was stirred overnight at room temperature. The solvent was removed under reduced pressure. Saturated aqueous NH₄Cl (5 mL) and DCM (5 mL) were added to the vial, and the resulting mixture was passed through a phase separator. The organic phase was concentrated under reduced pressure. The resulting product was dried under high vacuum for 30 minutes and then was dissolved in EtOAc (1 mL), and 4 M HCl in dioxane (1.1 mL) was added dropwise. The solution was stirred for 15 minutes at room temperature. Diethyl ether was added to induce the precipitation of a solid, which was filtered and washed with small portions of diethyl ether to afford the title compound (310 mg, 93% yield) was obtained as a white solid: ¹H NMR (400 MHz, Methanol-d₄) δ 7.62 (td, J=8.5, 6.2 Hz, 1H), 7.22-7.08 (m, 2H), 4.28 (d, J=1.3 Hz, 2H), 2.76 (s, 3H) (NH not observed).

### Example 18: Preparation of (E)-2-fluorobenzaldehyde O-methyl oxime

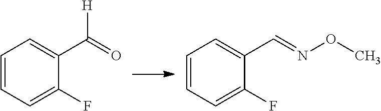

To a round-bottom flask equipped with a stir bar was added 2-fluorobenzaldehyde (0.849 mL, 8.06 mmol). The flask was evacuated and backfilled with nitrogen (3×). Anhydrous MeOH (16.1 mL) was added followed by pyridine (0.717 mL, 8.86 mmol). The septum was quickly removed, and O-methylhydroxylamine hydrochloride (0.673 g, 8.06 mmol) was added. The reaction mixture was stirred at room temperature overnight. UPLC indicated formation of both the major (E)-stereoisomer and minor (Z)-stereoisomer. The reaction mixture was poured into water (10 mL) and diluted further with DCM (20 mL). The biphasic mixture was passed through a phase separator and concentrated to afford a colorless oil. The resulting material was purified via flash column chromatography (SiO₂, 0→30% ethyl acetate in hexanes) to afford the title compound (969 mg, 78% yield) as a colorless oil: Product contained less than 3% of (Z)-oxime isomer: ¹H NMR (500 MHz, CDCl₃) δ 8.31 (s, 1H), 7.82 (td, J=7.6, 1.8 Hz, 1H), 7.37-7.31 (m, 1H), 7.17-7.10 (m, 1H), 7.07 (ddd, J=10.5, 8.3, 1.1 Hz, 1H), 3.99 (s, 3H); ¹³C NMR (126 MHz, CDCl₃) δ 160.92 (d, J=251.8 Hz), 142.38 (d, J=4.5 Hz), 131.45 (d, J=8.3 Hz), 126.89 (d, J=2.8 Hz), 124.45 (d, J=3.7 Hz), 120.22 (d, J=10.3 Hz), 115.97 (d, J=20.4 Hz), 62.34; EIMS m z 154 ([M+H]⁺).

### Example 19: Preparation of N-(2-fluorobenzyl)-O-methylhydroxylamine

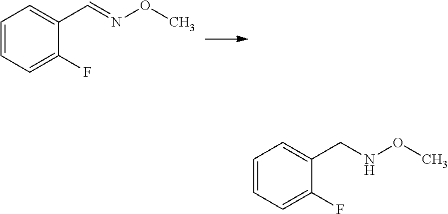

To a round bottom flask equipped with a stir bar was added (E)-2-fluorobenzaldehyde O-methyl oxime (969 mg, 6.33 mmol), indicator sodium (E)-4-((4-(dimethylamino)phenyl)diazenyl)benzenesulfonate (yellow above pH 4.4, red below pH 3.1; 20.7 mg, 0.0630 mmol), and anhydrous MeOH (24.5 mL) Sodium cyanoborohydride (1.99 g, 31.6 mmol) was added in portions, followed by 1 M HCl (0.192 mL, 6.33 mmol) until the solution went from orange/yellow to red. The reaction mixture was stirred at room temperature, and 1 M HCl was added periodically to maintain an acidic solution as the solution would go from red back to yellow/orange. After 1 h, UPLC showed consumption of starting material. The red-pink solution was diluted with saturated aqueous NaHCO₃ (15 mL) and DCM (25 mL). The mixture was passed through a phase separator and concentrated under reduced pressure (300 mbar, 28° C., the product was volatile) to afford the title compound (899 mg, 92% yield) as a yellow oil: ¹H NMR (500 MHz, CDCl₃) δ 7.37 (td, J=7.5, 1.8 Hz, 1H), 7.32-7.21 (m, 1H), 7.12 (td, J=7.5, 1.2 Hz, 1H), 7.05 (ddd, J=9.6, 8.2, 1.2 Hz, 1H), 5.78 (s, 1H), 4.12 (s, 2H), 3.52 (s, 3H); ¹⁹F NMR (471 MHz, CDCl₃) δ-119.30 (dt, J=12.3, 6.1 Hz); ESIMS m z 156 ([M+H]⁺).

### Example 20: Preparation of 2,5-dimethyl-N-(2-methylbenzyl)-4-nitrobenzamide

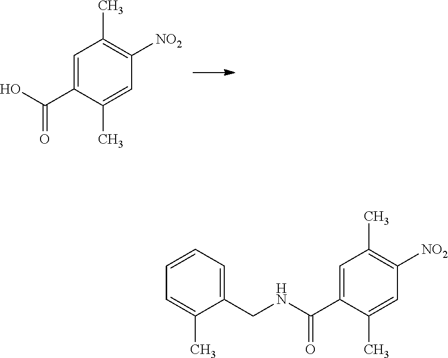 [SMILES: Cc1cc([N+](=O)[O-])c(C)cc1C(=O)O ; Cc1ccccc1CNC(=O)c1cc(C)c([N+](=O)[O-])cc1C]

In a small vial equipped with a stirbar, a solution of 2,5-dimethyl-4-nitrobenzoic acid (240 mg, 1.23 mmol), DMAP (45.1 mg, 0.369 mmol), and EDCI (354 mg, 1.85 mmol) was prepared in DCM (4.92 mL). o-Tolylmethanamine (0.305 mL, 2.46 mmol) was added in one portion via syringe. The resulting solution was allowed to stir overnight at ambient temperature. UPLC analysis indicated consumption of starting material. The solution was diluted with DCM (20 mL) and H₂O (20 mL). The biphasic mixture was passed through a phase separator and concentrated to afford an oil. The resulting material was purified by flash column chromatography (Cis Reverse phase, 30→100% acetonitrile in water) to afford the title compound (245 mg, 67% yield) as a white solid: ¹H NMR (500 MHz, CDCl₃) δ 7.82 (s, 1H), 7.33-7.28 (m, 2H), 7.25-7.18 (m, 3H), 5.85 (s, 1H), 4.64 (d, J=5.4 Hz, 2H), 2.55 (s, 3H), 2.48 (s, 3H), 2.40 (s, 3H); ¹³C NMR (126 MHz, CDCl₃) δ 167.86, 149.48, 140.67, 136.68, 135.58, 135.26, 131.22, 131.10, 130.95, 128.99, 128.39, 126.96, 126.59, 42.44, 19.99, 19.35, 19.24; ESIMS m/z 297 ([M−H])⁻).

### Example 21: Preparation of 2,5-dimethyl-N-(2-methylbenzyl)-4-nitro-N-(trifluoromethyl)benzamide

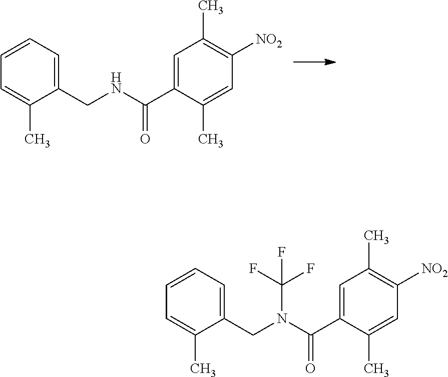 [SMILES: Cc1ccccc1CNC(=O)c1cc(C)c([N+](=O)[O-])cc1C ; Cc1ccccc1CN(C(=O)c1cc(C)c([N+](=O)[O-])cc1C)C(F)(F)F]

The installation of the N-trifluormethyl substituent followed the procedure reported in: Zhang, Z; He, J.; Zhu, L; Xiao, H.; Fang, Y.; Li, C. Chin. J. Chem. 2020, 38, 924-928. Cesium fluoride (624 mg, 4.11 mmol) was added to a 25 mL round-bottomed flask, placed under vacuum, and heated to 100° C. for 2 h. After cooling to ambient temperature, 1-(chloromethyl)-4-fluoro-1,4-diazabicyclo[2.2.2]octane-1,4-diium tetrafluoroborate (1.16 g, 3.28 mmol) and silver(I) trifluoromethanesulfonate (232 mg, 0.903 mmol) were added, and the flask was evacuated and backfilled with nitrogen gas (3×). 2,5-dimethyl-N-(2-methylbenzyl)-4-nitrobenzamide (245 mg, 0.821 mmol) was added dropwise as a solution in 3:1 DCM:phenyl chloride (16.4 mL), followed by 2-fluoropyridine (0.0780 mL, 0.903 mmol) and trimethyl(trifluoromethyl)silane (0.606 mL, 4.11 mmol). The reaction mixture was stirred vigorously at room temperature for 45 h. The reaction mixture was diluted with DCM (20 mL), and the reaction was quenched with H₂O (˜0.5 mL) until the solution became transparent. The reaction mixture was passed through a phase separator and concentrated to a yellow oil. The resulting material was purified by flash column chromatography (SiO₂, 0→50% 3:1 ethyl acetate:ethanol in hexanes) to afford the title compound (74.6 mg, 25% yield) as a yellow tinted oil: ¹H NMR (500 MHz, CDCl₃) δ 7.85 (s, 1H), 7.31-7.26 (m, 2H), 7.26-7.17 (m, 2H), 7.15 (s, 1H), 4.88 (d, J=2.1 Hz, 2H), 2.53 (s, 3H), 2.38 (s, 3H), 2.30 (s, 3H); ¹⁹F NMR (471 MHz, CDCl₃) δ-52.02; ESIMS m z 365 ([M−H]⁻).

### Example 22: Preparation of 4-amino-2,5-dimethyl-N-(2-methylbenzyl)-N-(trifluoromethyl)benzamide

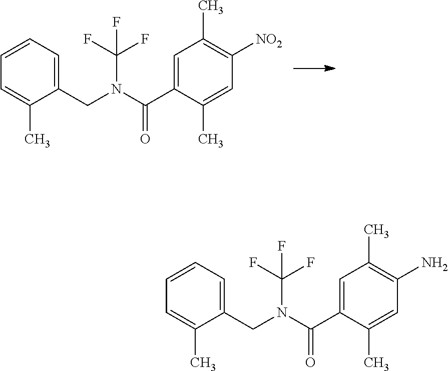 [SMILES: Cc1ccccc1CN(C(=O)c1cc(C)c([N+](=O)[O-])cc1C)C(F)(F)F ; Cc1cc(C(=O)N(Cc2ccccc2C)C(F)(F)F)c(C)cc1N]

In a small vial equipped with a stir bar, a mixture of 2,5-dimethyl-N-(2-methylbenzyl)-4-nitro-N-(trifluoromethyl)benzamide (70.0 mg, 0.191 mmol) and 5% palladium on carbon (40.7 mg, 0.019 mmol) was prepared in EtOAc (2.00 mL). The vial was fitted with a hydrogen balloon and was evacuated and filled with hydrogen gas (H₂, 3×). The reaction mixture was stirred at room temperature under an atmosphere of H₂ for 3 h. UPLC indicated consumption of starting material. The reaction solution was directly loaded onto Celite® and filtered using EtOAc. The organic phase was concentrated to afford the title compound (58.7 mg, 91% yield) as an orange oil that was used directly in the next step without further purification: ¹H NMR (500 MHz, CDCl₃) δ 7.30 (d, J=7.5 Hz, 1H), 7.26-7.11 (m, 3H), 6.94 (s, 1H), 6.48 (s, 1H), 4.80-4.75 (m, 2H), 3.74 (s, 2H), 2.26 (s, 3H), 2.23 (s, 3H), 2.05 (s, 3H); ¹⁹F NMR (471 MHz, CDCl₃) δ-52.83; ESIMS m z 337 ([M+H]⁺).

### Example 23: Preparation of tert-butyl (4-(difluoromethyl)benzyl)carbamate

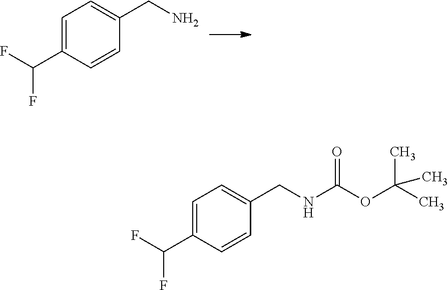 [SMILES: NCc1ccc(C(F)F)cc1 ; CC(C)(C)OC(=O)NCc1ccc(C(F)F)cc1]

In a small vial equipped with a stir bar, a solution of (4-(difluoromethyl)phenyl)methanamine (629 mg, 4.00 mmol) was prepared in DCM (3.00 mL). Di-tert-butyl dicarbonate (1.05 g, 4.80 mmol) and triethylamine (0.836 mL, 6.00 mL) were then added dropwise as a solution in DCM (5.00 mL), and the resulting solution was stirred at room temperature for 2 h. Brine (7 mL) was then added to the reaction mixture, and the biphasic mixture was passed through a phase separator. The organic phase was concentrated, and the resulting material was purified by flash column chromatography (SiO₂, 0→10% ethyl acetate in hexanes) to afford the title compound (900 mg, 87% yield) as white solid: ¹H NMR (400 MHz, CDCl₃) δ 7.47 (d, J=7.9 Hz, 2H), 7.36 (d, J=7.9 Hz, 2H), 6.83-6.41 (m, 1H), 4.92 (s, 1H), 4.35 (d, J=6.1 Hz, 2H), 1.46 (s, 9H); ¹⁹F NMR (376 MHz, CDCl₃) δ-110.35.

### Example 24: Preparation of tert-butyl (4-(difluoromethyl)benzyl)(methyl)carbamate

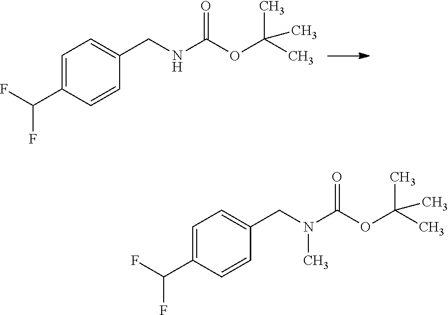 [SMILES: CC(C)(C)OC(=O)NCc1ccc(C(F)F)cc1 ; CN(Cc1ccc(C(F)F)cc1)C(=O)OC(C)(C)C]

In a small vial equipped with a stir bar, a solution of tert-butyl (4-(difluoromethyl)benzyl)carbamate (720 mg, 2.80 mmol) was prepared in dry THF (10.0 mL) and was cooled to 0° C. in an ice water bath. Lithium bis(trimethylsilyl)amide (1 M in THF, 4.6 mL, 3.64 mmol) was added dropwise via syringe. The ice bath was removed, and the solution was stirred for 2 h at room temperature. Iodomethane (0.226 mL, 3.64 mmol) was then added dropwise via syringe, and the resulting solution was stirred overnight at room temperature. The solvent was removed under reduced pressure. Brine (7 mL) and DCM (7 mL) were added, and the biphasic mixture was passed through a phase separator. The organic phase was concentrated. The resulting material was purified by flash column chromatography (SiO₂, 0→10% ethyl acetate in hexanes) to afford the title compound (508 mg, 67% yield) as a yellow oil: ¹H NMR (400 MHz, CDCl₃) δ 7.48 (d, J=7.8 Hz, 2H), 7.31 (d, J=7.7 Hz, 2H), 6.84-6.45 (m, 1H), 4.46 (s, 2H), 2.93-2.72 (m, 3H), 1.48 (d, J=10.5 Hz, 9H); ¹⁹F NMR (376 MHz, CDCl₃) δ-110.29.

### Example 25: Preparation of 1-(4-(difluoromethyl)phenyl)-N-methylmethanamine hydrochloride

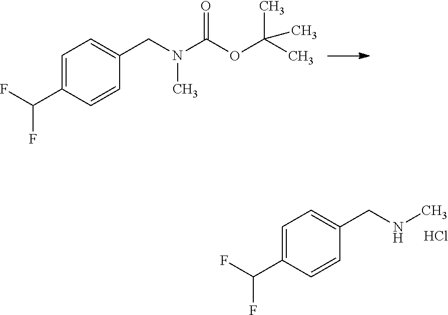 [SMILES: CN(Cc1ccc(C(F)F)cc1)C(=O)OC(C)(C)C ; CNCc1ccc(C(F)F)cc1 ; Cl]

In a small vial equipped with a stir bar, a solution of tert-butyl (4-(difluoromethyl)benzyl)(methyl)carbamate (488 mg, 1.799 mmol) was prepared in THF (5.00 mL). 4 M HCl in 1,4-dioxane (2.4 mL) was added dropwise via syringe. The vial was fitted with a reflux condenser, and the solution was stirred at 65° C. for 2 h. The reaction mixture was concentrated under a stream of nitrogen. Diethyl ether was added to afford a precipitate that was collected by filtration and washed with small portions of cold diethyl ether to afford the title compound (359 mg, 96% yield) as a white solid: ¹H NMR (500 MHz, DMSO-d₆) δ 9.54 (s, 2H), 7.71 (d, J=7.9 Hz, 2H), 7.63 (d, J=7.9 Hz, 2H), 7.29-6.89 (m, 1H), 4.16 (s, 2H), 2.52 (s, 3H); ¹⁹F NMR (471 MHz, DMSO-d₆) δ-109.82 (d, J=55.6 Hz); ESIMS m/z 172 ([M−Cl]⁺).

### General Biological Experimental Details

### Example A: Evaluation of Fungicidal Activity: Leaf Blotch of Wheat (Zymoseptoria Tritici; Bayer Code SEPTTR)

Technical grades of materials were dissolved in acetone, which were then mixed with nine volumes of water (H₂O) containing 110 ppm Triton X-100. The fungicide solutions were applied onto wheat seedlings using an automated booth sprayer to run-off. All sprayed plants were allowed to air dry prior to further handling. All fungicides were evaluated using the aforementioned method for their activity vs. all target diseases, unless stated otherwise.

Wheat plants (variety ‘Yuma’) were grown from seed in a greenhouse in soil-less potting mix until the first leaf was fully emerged, with 7-10 seedlings per pot. These plants were inoculated with an aqueous spore suspension of Zymoseptoria tritici either 3 days prior to fungicide treatment (3 day curative; 3DC) or 1 day after fungicide treatment (1 day protectant; 1DP). After inoculation the plants were kept in 100% relative humidity for three days to permit spores to germinate and infect the leaf. The plants were then transferred to a greenhouse for disease to develop. When disease symptoms were fully expressed on the 1st leaves of untreated plants, infection levels were assessed on a scale of 0 to 100 percent disease severity. Percent disease control was calculated using the ratio of disease severity on treated plants relative to untreated plants.

### Example B: Evaluation of Fungicidal Activity: Wheat Brown Rust (Puccinia Triticina; Synonym: Puccinia Reconditaf sp. Tritici; Bayer Code PUCCRT)

Wheat plants (variety ‘Yuma’) were grown from seed in a greenhouse in soil-less potting mix until the first leaf was fully emerged, with 7-10 seedlings per pot. These plants were inoculated with an aqueous spore suspension of Puccinia triticina after fungicide treatments. After inoculation, the plants were kept in a dark dew room with 100% relative humidity overnight to permit spores to germinate and infect the leaf. The plants were then transferred to a greenhouse for disease to develop. Fungicide formulation, application and disease assessment followed the procedures as described in Example A.

### Example C: Evaluation of Fungicidal Activity: Asian Soybean Rust (Phakopsora pachyrhizi; Bayer Code PHAKPA)

Technical grades of materials were dissolved in acetone, which were then mixed with nine volumes of H₂O containing 0.011% Tween-20. The fungicide solutions were applied onto soybean seedlings using an automated booth sprayer to run-off. All sprayed plants were allowed to air dry prior to further handling.

Soybean plants (variety ‘Williams 82’) were grown in soil-less potting mix, with one plant per pot. Ten-day-old seedlings were used for testing. Plants were inoculated as described in Example A. Plants were incubated for 24 h in a dark dew room with 100% relative humidity then transferred to a growth room for disease to develop. Fungicide formulation and application were made as described in Example A. When disease symptoms were fully expressed, disease severity was assessed on the sprayed leaves on a scale of 0 to 100 percent. Percent disease control was calculated using the ratio of disease severity on treated plants relative to untreated plants.

### Example D: Evaluation of Fungicidal Activity: Leaf Blotch of Barley (Rhynchosporium Commune; Bayer Code RHYNSE)

Barley plants (variety ‘Harrington’) were grown from seed in a greenhouse in soil-less potting mix until the first leaf was fully emerged, with 7-10 seedlings per pot. These plants were inoculated with an aqueous spore suspension of Rhynchosporium commune after fungicide treatments. After inoculation, the plants were kept in a dark dew room with 100% relative humidity for two days to permit spores to germinate and infect the leaf. The plants were then transferred to a greenhouse for disease to develop. Fungicide formulation and application were made as described in Example A. Disease assessment was conducted as described in Example A.

### Example E: Evaluation of Fungicidal Activity: Spot Blotch of Barley (Cochliobolus sativus; Bayer Code COCHSA)

Barley seedlings (variety ‘Harrington’) were propagated in soil-less potting mix, with each pot having 8 to 12 plants, and used for testing when the first leaf was fully emerged. Test plants were inoculated with a spore suspension of Cochliobolus sativus 24 h after fungicide treatments. After inoculation the plants were kept in 10000 relative humidity for two days to permit spores to germinate and infect the leaf. The plants were then transferred to a greenhouse for disease to develop. Fungicide formulation, application and disease assessment followed the procedures as described in Example A.

Lengthy table referenced here
US20240023549A1-20240125-T00001
Please refer to the end of the specification for access instructions.

Lengthy table referenced here
US20240023549A1-20240125-T00002
Please refer to the end of the specification for access instructions.

Lengthy table referenced here
US20240023549A1-20240125-T00003
Please refer to the end of the specification for access instructions.

Lengthy table referenced here
US20240023549A1-20240125-T00004
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20240023549A1). An electronic copy of the table will also be available from the USPTO upon request and payment of the fee set forth in 37 CFR 1.19(b)(3).

1. A compound of Formula I: 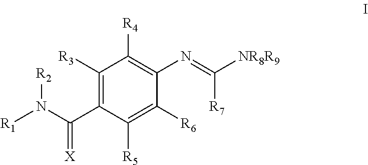
wherein
each R₁ and R₂ independently is selected from the group consisting of C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₃-C₈ cycloalkyl, C₃-C₈ substituted cycloalkyl, C₁-C₈ alkoxy, C₁-C₈ substituted alkoxy, C₃-C₈ heterocycloalkyl, C₃-C₈ substituted heterocycloalkyl, C₅-C₇ heteroaryl, C₅-C₇ substituted heteroaryl, aryl, substituted aryl, C₁-C₈ alkylaryl, substituted C₁-C₈ alkylaryl, C₁-C₈ alkyl(C₃-C₈ cycloalkyl), substituted C₁-C₈ alkyl(C₃-C₈ cycloalkyl), C₁-C₈ alkyl(C₃-C₈ heterocycloalkyl), substituted C₁-C₈ alkyl(C₃-C₈ heterocycloalkyl), C₁-C₈ alkyl(C₅-C₇ heteroaryl), and substituted C₁-C₈ alkyl(C₅-C₇ heteroaryl);
or R₁ and R₂ may be covalently bonded together to form a C₃-C₈ heterocycloalkyl, C₃-C₈ substituted heterocycloalkyl, a C₃-C₁₂ heteroaryl, or a C₃-C₁₂ substituted heteroaryl group;
each R₃, R₄, R₅, and R₆ independently is selected from the group consisting of hydrogen, halogen, cyano, nitro, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₁-C₈ alkoxy, and C₁-C₈ substituted alkoxy;
R₇ is selected from the group consisting of hydrogen, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₁-C₈ alkoxy, and C₁-C₈ substituted alkoxy;
or R₇ and R₈ may be covalently bonded together to form a C₃-C₈ heterocycloalkyl or C₃-C₈ substituted heterocycloalkyl group;
each R₈ and R₉ independently is selected from the group consisting of C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₃-C₈ cycloalkyl, C₃-C₈ substituted cycloalkyl, aryl, substituted aryl, C₁-C₈ alkylaryl, and substituted C₁-C₈ alkylaryl;
or R₈ and R₉ may be covalently bonded together to form a saturated or unsaturated C₃-C₈ heterocycloalkyl or C₃-C₈ substituted heterocycloalkyl group;
X is selected from the group consisting of O and S;
wherein any and all heterocyclic rings may contain up to three heteroatoms selected from the group consisting of O, N, and S;
or a tautomer or salt thereof.

2. The compound of claim 1, wherein each R₁ and R₂ independently is selected from the group consisting of C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₃-C₈ cycloalkyl, C₃-C₈ substituted cycloalkyl, C₁-C₈ alkoxy, C₁-C₈ substituted alkoxy, aryl, substituted aryl, C₁-C₈ alkylaryl, and substituted C₁-C₈ alkylaryl.

3. The compound of claim 1, wherein R₁ is selected from the group consisting of C₁-C₈ alkylaryl and substituted C₁-C₈ alkylaryl.

4. The compound of claim 1, wherein R₂ is selected from the group consisting of C₁-C₈ alkyl, C₁-C₈ substituted alkyl, C₂-C₈ alkenyl, C₂-C₈ substituted alkenyl, C₁-C₈ alkoxy, and C₁-C₈ substituted alkoxy.

5. The compound of claim 1, wherein R₃ and R₆ are both hydrogen.

6. The compound of claim 5, wherein each R₄ and R₈ independently is selected from the group consisting of halogen, C₁-C₈ alkyl, C₁-C₈ substituted alkyl, and C₁-C₈ alkoxy.

7. The compound of claim 6, wherein R₄ and R₅ are both CH₃.

8. The compound of claim 1, wherein R₅ and R₆ are both hydrogen.

9. The compound of claim 8, wherein each R₃ and R₄ independently is selected from the group consisting of halogen, C₁-C₈ alkyl, C₁-C₈ substituted alkyl, and C₁-C₈ alkoxy.

10. The compound of claim 9, wherein R₃ and R₄ are both CH₃.

11. The compound of claim 1, wherein each R₈ and R₉ independently is selected from the group consisting of C₁-C₈ alkyl, C₁-C₈ substituted alkyl, C₂-C₈ alkenyl, C₃-C₈ cycloalkyl, aryl, substituted aryl, C₁-C₈ alkylaryl, and substituted C₁-C₈ alkylaryl.

12. The compound of claim 1, wherein R₇ is selected from the group consisting of hydrogen, C₁-C₈ alkyl, and C₁-C₈ substituted alkyl.

13. The compound of claim 1, wherein X is O.

14. A compound, wherein the compound is selected from one of the compounds in Table 1.

15. A fungicidal compound of Formula I: 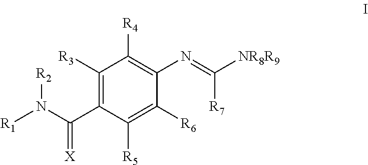
wherein
each R₁ and R₂ independently is selected from the group consisting of C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₃-C₈ cycloalkyl, C₃-C₈ substituted cycloalkyl, C₁-C₈ alkoxy, C₁-C₈ substituted alkoxy, C₃-C₈ heterocycloalkyl, C₃-C₈ substituted heterocycloalkyl, C₅-C₇ heteroaryl, C₅-C₇ substituted heteroaryl, aryl, substituted aryl, C₁-C₈ alkylaryl, substituted C₁-C₈ alkylaryl, C₁-C₈ alkyl(C₃-C₈ cycloalkyl), substituted C₁-C₈ alkyl(C₃-C₈ cycloalkyl), C₁-C₈ alkyl(C₃-C₈ heterocycloalkyl), substituted C₁-C₈ alkyl(C₃-C₈ heterocycloalkyl), C₁-C₈ alkyl(C₅-C₇ heteroaryl), and substituted C₁-C₈ alkyl(C₅-C₇ heteroaryl);
or R₁ and R₂ may be covalently bonded together to form a C₃-C₈ heterocycloalkyl, C₃-C₈ substituted heterocycloalkyl, a C₃-C₁₂ heteroaryl, or a C₃-C₁₂ substituted heteroaryl group;
each R₃, R₄, R₅, and R₆ independently is selected from the group consisting of hydrogen, halogen, cyano, nitro, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₁-C₈ alkoxy, and C₁-C₈ substituted alkoxy;
R₇ is selected from the group consisting of hydrogen, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C alkoxy, C₁-C₈ substituted alkoxy, and thiol;
or R₇ and R₈ may be covalently bonded together to form a C₃-C₈ heterocycloalkyl or C₃-C₈ substituted heterocycloalkyl group;
each R₈ and R₉ independently is selected from the group consisting of C₁-C₈ alkyl, C₁-C₈ substituted alkyl, C₂-C₈ alkenyl, C₂-C₈ substituted alkenyl, C₂-C₈ alkynyl, C₃-C₈ substituted alkynyl, C₃-C₈ cycloalkyl, C₃-C₈ substituted cycloalkyl, aryl, substituted aryl, C₁-C₈ alkylaryl, and substituted C₁-C₈ alkylaryl;
or R₈ and R₉ may be covalently bonded together to form a saturated or unsaturated C₃-C₈ heterocycloalkyl or C₃-C₈ substituted heterocycloalkyl group;
X is selected from the group consisting of O and S;
wherein any and all heterocyclic rings may contain up to three heteroatoms selected from the group consisting of O, N, and S;
or a tautomer or salt thereof, for controlling a fungal pathogen.

16. The fungicidal compound of claim 15, wherein the fungal pathogen is one of Zymoseptoria tritici, Cochliobolus sativus, Puccinia triticina, Puccinia striiformis, Venturia inaequalis, Ustilago maydis, Uncinula necator, Rhynchosporium commune, Magnaporthe grisea, Phakopsora pachyrhizi, Parastagonospora nodorum, Glomerella lagenarium, Cercospora beticola, Alternaria solani, Pyrenophora teres, Blumeria graminis f. sp. tritici, Blumeria graminis f. sp. hordei, Erysiphe cichoracearum, Fusarium virguliforme, Rhizoctonia solani, Pythium ultimum, Botrytis cinerea, Ramularia collo-cygni, Pyrenophora tritici-repentis, Exserohilum turcicum, Puccinia polysora, Sclerotinia sclerotiorum, Erysiphe diffusa, Fusarium graminearum, Podosphaera leucotricha, Colletotrichum truncatum, Cercospora kikuchii, Cerospora sojina, Corynespora cassiicola, and Septoria glycines.

17. The fungicidal compound of claim 15, wherein the compound treats one of the following diseases from the fungal pathogen: Septoria Leaf Blotch of Wheat (Zymoseptoria tritici), Spot Blotch of Barley (Cochliobolus sativus), Wheat Brown Rust (Puccinia triticina), Stripe Rust of Wheat (Puccinia striiformis), Scab of Apple (Venturia inaequalis), Blister Smut of Maize (Ustilago maydis), Powdery Mildew of Grapevine (Uncinula necator), Leaf Blotch of Barley (Rhynchosporium commune), Blast of Rice (Magnaporthe grisea), Asian Soybean Rust (Phakopsora pachyrhizi), Glume Blotch of Wheat (Parastagonospora nodorum), Anthracnose of Cucurbits (Glomerella lagenarium), Leaf Spot of Beet (Cercospora beticola), Early Blight of Tomato (Alternaria solani), Net Blotch of Barley (Pyrenophora teres), Powdery Mildew of Wheat (Blumeria graminis f. sp. tritici), Powdery Mildew of Barley (Blumeria graminis f. sp. hordei), Powdery Mildew of Cucurbits (Erysiphe cichoracearum), Sudden Death Syndrome of Soybean (Fusarium virguliforme), Collar Rot or Damping-Off of Seedlings (Rhizoctonia solani), Root Rot (Pythium ultimum), Grey Mold (Botrytis cinerea), Ramularia Leaf Spot (Ramularia collo-cygni), Tan Spot of Wheat (Pyrenophora tritici-repentis), Northern Leaf Blight of Maize (Exserohilum turcicum), Southern Rust of Maize (Puccinia polysora), White Mold (Sclerotinia sclerotiorum), Powdery Mildew of Soybean (Erysiphe diffusa), Head Blight of Cereals (Fusarium graminearum), Powdery Mildew of Apple (Podosphaera leucotricha), Anthracnose of Soybean (Colletotrichum truncatum), Cercospora Leaf Blight (Cercospora kikuchii), Frogeye Leaf Spot (Cerospora sojina), Target Spot of Soybean (Corynespora cassiicola), Leaf Spot of Soybean (Septoria glycines).

18. A composition for use in the control of a fungal pathogen, the composition comprising a phytologically acceptable amount of the compound of claim 1 and a carrier.

19. The composition of claim 18, wherein the carrier is one or more of a thickener, emulsifier, rheology agent, dispersant and polymer.

20. The composition of claim 18, wherein the fungal pathogen is one of Zymoseptoria tritici, Cochliobolus sativus, Puccinia triticina, Puccinia striiformis, Venturia inaequalis, Ustilago maydis, Uncinula necator, Rhynchosporium commune, Magnaporthe grisea, Phakopsora pachyrhizi, Parastagonospora nodorum, Glomerella lagenarium, Cercospora beticola, Alternaria solani, Pyrenophora teres, Blumeria graminis f. sp. tritici, Blumeria graminis f. sp. hordei, Erysiphe cichoracearum, Fusarium virguliforme, Rhizoctonia solani, Pythium ultimum, Botrytis cinerea, Ramularia collo-cygni, Pyrenophora tritici-repentis, Exserohilum turcicum, Puccinia polysora, Sclerotinia sclerotiorum, Erysiphe diffusa, Fusarium graminearum, Podosphaera leucotricha, Colletotrichum truncatum, Cercospora kikuchii, Cerospora sojina, Corynespora cassiicola, and Septoria glycines.

21. The composition of claim 18, wherein the composition treats one of the following diseases from the fungal pathogen: Septoria Leaf Blotch of Wheat (Zymoseptoria tritici), Spot Blotch of Barley (Cochliobolus sativus), Wheat Brown Rust (Puccinia triticina), Stripe Rust of Wheat (Puccinia striiformis), Scab of Apple (Venturia inaequalis), Blister Smut of Maize (Ustilago maydis), Powdery Mildew of Grapevine (Uncinula necator), Leaf Blotch of Barley (Rhynchosporium commune), Blast of Rice (Magnaporthe grisea), Asian Soybean Rust (Phakopsora pachyrhizi), Glume Blotch of Wheat (Parastagonospora nodorum), Anthracnose of Cucurbits (Glomerella lagenarium), Leaf Spot of Beet (Cercospora beticola), Early Blight of Tomato (Alternaria solani), Net Blotch of Barley (Pyrenophora teres), Powdery Mildew of Wheat (Blumeria graminis f. sp. tritici), Powdery Mildew of Barley (Blumeria graminis f. sp. hordei), Powdery Mildew of Cucurbits (Erysiphe cichoracearum), Sudden Death Syndrome of Soybean (Fusarium virguliforme), Collar Rot or Damping-Off of Seedlings (Rhizoctonia solani), Root Rot (Pythium ultimum), Grey Mold (Botrytis cinerea), Ramularia Leaf Spot (Ramularia collo-cygni), Tan Spot of Wheat (Pyrenophora tritici-repentis), Northern Leaf Blight of Maize (Exserohilum turcicum), Southern Rust of Maize (Puccinia polysora), White Mold (Sclerotinia sclerotiorum), Powdery Mildew of Soybean (Erysiphe diffusa), Head Blight of Cereals (Fusarium graminearum), Powdery Mildew of Apple (Podosphaera leucotricha), Anthracnose of Soybean (Colletotrichum truncatum), Cercospora Leaf Blight (Cercospora kikuchii), Frogeye Leaf Spot (Cerospora sojina), Target Spot of Soybean (Corynespora cassiicola), Leaf Spot of Soybean (Septoria glycines).

22. The composition of claim 21, wherein the disease is one of Septoria Leaf Blotch of Wheat, Spot Blotch of Barley, Leaf Blotch of Barley, Wheat Brown Rust, and Asian Soybean Rust.

23. A seed treated with a phytologically acceptable amount of the compound of claim 1.

24. A method of controlling fungal attack on a plant, the method comprising contacting an area adjacent to the plant, soil adapted to support growth of the plant, a root of the plant, and foliage of the plant, with a phytologically acceptable amount of the compound of claim 1.

25. A seed treated with a phytologically acceptable amount of the composition of claim 18.

26. A method of controlling fungal attack on a plant, the method comprising contacting an area adjacent to the plant, soil adapted to support growth of the plant, a root of the plant, and foliage of the plant, with a phytologically acceptable amount of the composition of claim 18.
